(19) United States
(12) Patent Application Publication
(10) Pub. No.: US 2023/0181691 A1
(43) Pub. Date: Jun. 15, 2023

(54) PLATELET FACTORS AND COGNITIVE IMPROVEMENT

(71) Applicant: The Regents of the University of California, Oakland, CA (US)

(72) Inventors: Dena Dubal, San Francisco, CA (US); Cana Park, Oakland, CA (US); Saul Villeda, San Francisco, CA (US); Adam Schroer, San Francisco, CA (US); Patrick Ventura, San Francisco, CA (US)

(73) Assignee: The Regents of the University of California, Oakland, CA (US)

(21) Appl. No.: 17/799,444

(22) PCT Filed: Feb. 11, 2021
(86) PCT No.: PCT/US2021/017580

§ 371 Date: Aug. 12, 2022

Related U.S. Application Data
(60) Provisional application No. 62/975,591, filed on Feb. 12, 2020.

(51) Int. Cl. A61K 38/19; A61P 25/28; A61K 9/00

(52) U.S. Cl. CPC A61K 38/19; A61P 25/28; A61K 9/0019

(57) ABSTRACT

Provided herein are Platelet Activating Factor 4 (PF4) polypeptide and other compositions and methods for improving cognitive function in an individual comprising treatment with PF4 and other polypeptides.

### CROSS-REFERENCE TO RELATED PATENT APPLICATIONS

The present application claims benefit of priority to U.S. Provisional Patent application No. 62/975,591, filed Feb. 12, 2020, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under grants no. OD012178 and RO1 AG053382 awarded by The National Institutes of Health. The government has certain rights in the invention.

### BACKGROUND OF THE INVENTION

Brain health is one of the biggest biomedical challenges with few if any effective medical treatments. Cognition is a highly valued and central manifestation of brain health that is impaired or becomes disrupted in normal aging, numerous neurodegenerative, neurologic, and psychiatric diseases, childhood developmental syndromes, traumatic brain injury, and stress. Cognition is also disrupted by jet lag, medication side effects, and certain medical treatments, such as those for cancer. Thus, the potential to enhance cognition or counter cognitive dysfunction is of enormous relevance across the human lifespan in health and disease.

### BRIEF SUMMARY OF THE INVENTION

The disclosure provides methods for improving cognitive function in an individual in need thereof. In some embodiments, the method comprises administering to the individual an effective amount of a protein comprising a polypeptide of Table 1 or Table 2 or a functional fragment or variant thereof, wherein the administering is systemic or peripheral, thereby improving cognitive function in the individual.

In some embodiments, the polypeptide comprises Platelet Activating Factor 4 (PF4) or a functional fragment or variant thereof. In some embodiments, the polypeptide comprises an amino acid sequence at least 70, 75, 80, 85, 90, 95, 97, or 99% identical to SEQ ID NO:1.

In some embodiments, the administering is oral, mucosal, or carried out by injection. In some embodiments, the injection is intravenous, intraperitoneal, subcutaneous, or intramuscular.

In some embodiments, the individual is a human. In some embodiments, the human has at least normal cognitive function and the administering results in improved cognitive function compared to before the administering. In some embodiments, the human is 50 years of age or older. In some embodiments, the human has age related cognitive decline. In some embodiments, the human is less than 50 years of age.

In some embodiments, the individual is a human having a neurodegenerative disease. In some embodiments, the neurodegenerative disease is selected from the group consisting of: Alzheimer's disease, Parkinson's disease, Huntington's disease, frontotemporal dementia, progressive supranuclear palsy, corticobasalar degeneration, mild cognitive impairment, vascular dementia, Lewy body dementia, multiple system atrophy, amyotropic lateral sclerosis, prion disorder, and HIV-related dementia.

In some embodiments, the individual is a human having a condition selected from the group consisting of: depression, schizophrenia, attention deficit/hyperactivity disorder, autism spectrum disorder, intellectual disability, a mood disorder, and a psychotic disorder.

In some embodiments, the individual is a human having a condition selected from the group consisting of traumatic brain injury, stroke, multiple sclerosis, neuroautoimmune disease, epilepsy, delirium, and a paraneoplastic disorder.

In some embodiments, the individual is a human having a condition selected from the group consisting of: an X-linked mental disorder, Down's syndrome, Angelman's syndrome, Rett's syndrome, phenylketonuria, Lesch-Nyhan, galactosemia, and adrenoleukodystrophy.

In some embodiments, the individual is a human having a condition selected from astrocytoma, ependymoma, medulloblastoma, and oligodendroglioma.

In some embodiments, the individual is a human receiving radiation treatment or chemotherapy for cancer.

In some embodiments, the individual is a human that is experiencing, or will experience within 24 hours, sleep deprivation or jet lag.

In some embodiments, the effective amount is 1 μg to 1000 μg per kg body weight of the individual.

In some embodiments, the polypeptide or a functional fragment thereof is administered more than once as part of a course of treatment. In some embodiments, the polypeptide or a functional fragment thereof is administered once every 1-7 days.

In some embodiments, the method further comprises testing the cognitive function of the individual after administering. In some embodiments, the method further comprises testing the cognitive function of the individual prior to administering, and comparing the cognitive function of the individual prior to and after administering.

In some embodiments, cognitive function is determined by testing the individual for semantic, episodic, procedural, priming, and/or working memory.

Also provided is a method for improving motor function or motor learning or both in an individual in need thereof. In some embodiments, the method comprises administering to the individual an effective amount of a protein comprising a polypeptide of Table 1 or Table 2 or a functional fragment or variant thereof, wherein the administering is systemic or peripheral, thereby improving motor function in the individual compared to before the administering.

### Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The terms “PF4” or “CXCL4” refer to human Platelet Activating Factor 4 polypeptide, and functional variants and fragments thereof, unless otherwise stated. A variant of PF4 is known as “CXCL4var” or CXCL4L1.” PF4 is expressed as a precursor protein of around 100 amino acids (see, e.g. SwissProt P02776) that is later processed into a mature protein of around 70 amino acids. An exemplary mature human PF4 polypeptide sequence is EAEEDGDLQCLCVKTTSQV RPRHITSLEV IKAGPHCPTAQLIATLKNGR KICLDLQAPL YKKIIKKLLES (SEQ ID NO:1) In some embodiments, the PF4 protein or functional fragment thereof comprises a heparin-binding site at the C-terminus comprising a sequence of KKIIKK. In other embodiments, that site is absent. The mature human PF4 protein comprises nine beta strands (at amino acid positions 38-40, 42-44, 55-61, 64-66, 67-79, 71-76, 77-79, 81-83, and 86-88) and two helix (52-54, and 90-99 amino acids). Functional variants or fragments can have all or fewer than nine of the beta strands (e.g., 8, 7, 6, 5, 4) and/or all or fewer (e.g., one or none) of the helices. In some embodiments, the PF4 protein comprises an amino terminal signal sequence. For example, in some embodiments, the PF4 protein comprises

(SEQ ID NO: 19)
MSSAAGFCASRPGLLFLGLLLLPLVVAFASAEAEEDGDL
QCLCVKTTSQVRPRHITSLEVIKAGPHCPTAQLIATLKN
GRKICLDLQAPLYKKIIKKLLES.

Some exemplary naturally-occurring variants of PF4 have one or more of the following amino acid changes (as measured from the mature protein): P58L, K66E, and/or L67H. Variants are described in, e.g., Kuo, et al., J. Biol. Chem. VOL. 288, NO. 19, pp. 13522-13533, May 10, 2013. PF4 binds the CXCR3 and CCR1 receptor. PF4 promotes blood coagulation by moderating the effects of heparin-like molecules.

As used herein, the terms “systemic” or “peripheral” refer to administration by a route that does not involve direct injection (or other administration) into the cerebrospinal fluid (CSF) or central nervous system (CNS). That is, systemic and peripheral administration encompasses administration to the “blood” side of the blood-brain barrier. Examples of systemic and peripheral routes include oral and mucosal, intravenous, intraperitoneal, intramuscular, and subcutaneous injection, and intravenous drip.

The terms “cognition,” “cognitive ability,” “cognitive function,” and like terms refer to a collection of mental tasks and functions, including but not limited to: memory (e.g., semantic, episodic, procedural, priming, or working); orientation; language; problem solving; visual perception, construction, and integration; planning; organizational skills; selective attention; inhibitory control; and ability to mentally manipulate information.

The terms “improved cognition,” “increased cognitive ability,” “improved cognitive function,” and like terms refer to an improvement in cognition under a given condition (e.g. treatment with a polypeptide as described herein) compared to cognition absent the condition (e.g., absent treatment with the polypeptide). For an individual experiencing cognitive decline, an improvement in cognition might be a reduction in the rate of cognitive decline (i.e., an improvement compared to the absence of treatment), but not an actual improvement in cognitive ability. An increase in cognitive ability can also be an increase in brain activity in a specified area, e.g., as determined by MRI, or an inhibition of brain activity that results in better overall brain function. An increase in cognitive ability can also be improvement in a cognitive performance test as described in more detail herein. An improvement or increase in cognitive ability can be in any one cognitive aspect or function, or any combination of individual cognitive functions.

An individual in need of improved cognitive function refers to individuals with age-related cognitive decline; a neurodegenerative disease; a mental or mood disorder; traumatic brain injury; developmental delay; genetic disorder resulting in reduced cognitive ability; brain injury due to stroke, brain cancer, MS, epilepsy, radiation or chemotherapy; etc. An individual in need of improved cognitive function can also include individuals that desire increased mental function to fight the effects of stress, sleep deprivation, jet lag, or pain, or to heighten ability for a particular task. A more complete and specific list of such individuals is included below.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the algorithms can account for gaps and the like. Typically, identity exists over a region comprising an antibody epitope, or a sequence that is at least about 25 amino acids or nucleotides in length, or over a region that is 50-100 amino acids or nucleotides in length, or over the entire length of the reference sequence.

Individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Such conservatively modified variants are in addition to and do not exclude polymorphic variants, interspecies homologs, and alleles of the invention. The following amino acids are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a protein or nucleic acid indicates that the protein or nucleic acid comprises two or more subsequences that are not found in the same relationship to each other in nature. For instance, the protein or nucleic acid is typically recombinantly produced, having two or more sequences from unrelated genes arranged to make a new functional nucleic acid, e.g., a promoter from one source and a coding region from another source, or functional chimeric protein.

The terms “agonist,” “activator,” “inducer” and like terms refer to an agent that increases activity or expression (e.g., of a protein of Table 1 or Table 2) as compared to a control. Agonists are agents that, e.g., stimulate, increase, activate, enhance activation, sensitize or upregulate the activity of a protein of Table 1 or Table 2. The expression or activity can be increased 10%, 20%, 30%, 40%, 50%, 60%, 70%, 80%, 90% 100% or more than that in a control. In certain instances, the activation is 1.5-fold, 2-fold, 3-fold, 4-fold, 5-fold, 10-fold, or more in comparison to a control.

The terms “inhibitor,” “repressor” or “antagonist” or “down regulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. Controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known for conjugating a protein to the label may be employed, e.g., using methods described in Hermanson, Bioconjugate Techniques 1996, Academic Press, Inc., San Diego.

The term “diagnosis” refers to a relative probability that a disorder is present in an individual. Similarly, the term “prognosis” refers to a relative probability that a certain future outcome may occur in the individual. For example, in the context of the present disclosure, prognosis can refer to the likelihood that an individual suffer cognitive decline, or the likely severity of the disease (e.g., severity of symptoms, rate of functional decline, etc.). The terms are not intended to be absolute, as will be appreciated by any one of skill in the field of medical diagnostics.

A “biological sample” can be obtained from a patient, e.g., a biopsy, from an animal, such as an animal model, or from cultured cells, e.g., a cell line or cells removed from a patient and grown in culture for observation. Biological samples include tissues and bodily fluids, e.g., cerebrospinal fluid (CSF), blood, blood fractions, lymph, saliva, urine, feces, etc.

The terms “therapy,” “treatment,” and “amelioration” refer to any reduction in the severity of symptoms (cognitive decline), or improvement in cognitive function, or where motor function is affected, an improvement in motor function. As used herein, the terms “treat” and “prevent” are not intended to be absolute terms. Treatment and prevention can refer to any delay in cognitive decline, amelioration of symptoms (e.g., confusion, delirium), etc. Treatment and prevention can be complete or partial, such that cognition is better than would be expected without treatment (e.g., compared to cognition in the same individual before treatment or compared to cognition in similar non-treated individuals). The effect of treatment can be compared to an individual or pool of individuals not receiving the treatment, or to the same patient prior to treatment or at a different time during treatment. In some aspects, cognition is improved by at least 1%, as compared, e.g., to the individual before administration or to a control individual not undergoing treatment. In some embodiments, cognition is improved by at least 2, 3, 5, 7, 10, 15, 20, 25%, 50%, 75%, 80%, or 90%, or more, determined using tests of cognition, molecular proxies, or structural changes associated with brain function. In some aspects, motor function is improved by at least 1%, as compared, e.g., to the individual before administration or to a control individual not undergoing treatment. In some embodiments, motor function is improved by at least 2, 3, 5, 7, 10, 15, 20, 25%, 50%, 75%, 80%, or 90%, or more, determined using tests of motor function.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least 1%, 2%, 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present disclosure, the dose refers to the amount of polypeptide selected from Table 1 or Table 2. The dose will vary depending on a number of factors, including frequency of administration; size and tolerance of the individual; type and severity of the condition; risk of side effects; and the route of administration. The dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as dogs, horses, pigs, mice, rats, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

### DESCRIPTION OF THE DRAWINGS

FIG. 1A-C: (1A) Paradigm for plasma proteomics profiling. Mice were injected with klotho, allowed to explore a small Y-maze for 10 minutes, and then their plasma was immediately harvested. (1B) Plasma proteomics identified PF4 as highest expressed klotho-induced protein (6.5 fold increase, FDR q-value=0.002). (1C) Pathway analysis predicts klotho activates platelets and their functions.

FIG. 2A-B: (2A) Paradigm of Veh or PF4 Trt in aging mice followed by cognitive testing (n=8 mice/group, age 18-21 mos). (2B) PF4 treatment given one hour before training and then 1 h before testing increased memory of aging mice, measured by time spent exploring the novel compared to familiar arms of the Two Trials Y-maze over several minutes (Two-way repeated measures ANOVA, *p<0.05). Data are mean±SEM.

FIG. 3A-B: Young plasma contains platelets. 3A, Illustration of centrifugation-based plasma collection and fractionation of the soluble and platelet-enriched fractions of plasma. Micrographs depict blood and plasma smears. Arrows indicate platelets within both samples. Plasma was collected from blood of young (2 months) male mice by centrifugation at 1,000 g for 10 min. Plasma was centrifuged at 20,000 g for 10 min. The supernatant was collected as the soluble fraction of plasma and the pelleted platelet-enriched fraction was resuspended in an equivalent volume of saline. 3B, Platelet enrichment was confirmed via Western blot analysis of the platelet marker, Thrombospondin-1 (THSB1).

FIG. 4A-C: Young plasma and the platelet-enriched fraction of plasma promote Creb activation in the aged hippocampus. 4A, Schematic illustrating the timeline of tail vein injection of 100 μL of young plasma, young soluble fraction, and young platelet-enriched fraction to aged male mice (20 months). 4B, Quantification of Creb phosphorylation (p-Creb) in the dentate gyrus of the hippocampus following specified treatment, and 4C, representative images. n=5-7 per group. Data represented as mean+/−SEM; one-way ANOVA with Dunnett's post-hoc test (B); **p<0.01, ***p<0.001.

FIG. 5A-D. Young plasma and the platelet-enriched fraction of plasma rejuvenate hippocampal-dependent cognitive function in aged mice. 5A, Schematic illustrating the timeline of tail vein injection of 100 μL of saline (n=11), young plasma (n=10), or the platelet-enriched fraction of young plasma (n=12) to aged male mice (20 months), followed by cognitive testing. 5B, Object recognition memory was assessed by Novel Object Recognition (NOR), as time spent exploring a novel object relative to a familiar object, 24 h after training 5C-5D, Associative fear memory was assessed using contextual (5C) and cued 5(D) fear conditioning, as percentage freezing time 24 h after training. Data shown as mean+/−s.e.m.; (5B) one-sample t-test vs 50%; (5C-5D) one-way ANOVA with Dunnett's post-hoc test; *p<0.05, **p<0.01, ***p<0.001.

FIG. 6A-G. The platelet-enriched fraction of young plasma mitigates inflammation in the aged hippocampus. Aged (20 months) male mice were administered 100 μL of saline, young plasma, or the platelet-enriched fraction of young plasma by tail vein injection, 9 times over a 24-day period. 6A, Heatmap of overlapping genes significantly differentially expressed in the hippocampus of aged (20 months) male mice following systemic administration of both young plasma and the platelet-enriched fraction of young plasma (n=5-6 per group). 6B, Top 10 significant Biological Processes Gene Ontology (GO) terms associated with genes upregulated in the hippocampus following both treatment with young plasma and the young platelet-enriched fraction of young plasma. 6C-6E, The expression of inflammatory-related genes (6C, Tnfa, 6D, Clq-b, and 6E, CD11b) were assessed in a separate cohort via qRT-PCR (n=6 per group). 6F-6G, Hippocampal microglial activation was analyzed by Ibal and CD68 immunostaining. 6F, Representative images and 6G, quantification of Ibal/CD68 double positive cells in the dentate gyrus of the aged hippocampus (n=10-12 per group). Data represented as mean+/−SEM; one-way ANOA with Dunnett's post-hoc test; *p<0.05.

FIG. 7A-D. Platelet Factor-4 (PF4) is elevated in young relative to aged platelet-enriched fraction of plasma. 7A, The top 10 proteins identified as most enriched in the platelet-enriched fraction of plasma isolated from young (2 months) male mice relative to old (20 months) male mice using proteomic mass spectrometry. 7B, Western blot analysis of Platelet Factor-4 (PF4) in the plasma, soluble fraction of plasma, and platelet-enriched fraction of plasma from young (2 months) male mice. 7C, Western blot analysis of PF4 in the platelet-enriched fraction of plasma from young (2 months) and aged (20 months) male mice. 7D, ELISA of PF4 in the plasma from young (2 months) and aged (20 months) male mice. n=8 per group. Data represented as mean+/−SEM; unpaired t-test; *p<0.05.

FIG. 8A-C. Systemic PF4 treatment promotes Creb activation in the aged hippocampus. 8A, Schematic illustrating the timeline of tail vein injection of 100 μL of saline or PF4 (5 μg/mL) to aged (20 months) male mice. 8B, Quantification of Creb phosphorylation (p-Creb) by immunolabeling in the hippocampus of aged (20 months) male mice following treatment, and 8C, representative images. n=6 per group. Data represented as mean+/−SEM; unpaired t-test (B); **p<0.01.

FIG. 9A-C. Systemic PF4 treatment rejuvenates hippocampal-dependent cognitive function in aged mice. 9A, Schematic illustrating the timeline of tail vein injection of 100 μL of saline or PF4 (5 μg/mL) to aged (20 months) male mice, followed by cognitive testing. 9B, Object recognition memory was assessed by Novel Object Recognition (NOR), and quantified as time spent exploring a novel object relative to a familiar object, 24 h after training. 9C, Hippocampal-dependent learning and memory was evaluated by radial arm water maze (RAWM). Changes in cognition were quantified as number of errors while attempting to find the goal arm. Data represented as mean+/−SEM; (9B) one-sample t-test vs 50%; (9C) ANOVA with Tukey's post-hoc.; *p<0.05, **p<0.01, **p<0.001.

FIG. 10A-C. Systemic PF4 treatment mitigates inflammation in the aged hippocampus. Aged (20 months) male mice were administered 100 μL of saline or PF4 (5 μg/mL) by tail vein injection, 9 times over a 24-day period. 10A, The expression of inflammatory-related genes (Tnfa, Nfkb, Il1b, C1q-b, CD11b, and C3) were assessed via qRT-PCR. 10B-10C, Hippocampal microglia were analyzed by Ibal and CD68 immunostaining 10B, Representative images and 10C, quantification of CD68 positive cells in the dentate gyrus of the aged hippocampus (n=6 per group). Data represented as mean+/−SEM; unpaired t-test; *p<0.05, **p<0.01.

FIG. 11A-C. PF4 mitigates LPS-induced expression of inflammatory cytokines in BV2 microglial cells. BV2 cells were treated with PF4 (100 ng/mL) or vehicle; after 60 min cells were stimulated with lipopolysaccharide (LPS; 200 ng/mL) or vehicle. 24 hours later RNA was extracted to assess transcript abundance of genes associated with inflammatory cytokines (11A, Tnfa, 11B, Nfkb, and 11C, Il1b). Data represented as mean+/−SEM; one-way ANOVA with Dunnett's post-hoc test; **p<0.01, ***p<0.001, ****p<0.0001.

FIG. 12. PF4 increases synaptic plasticity, a process that underlies learning and memory, in the CA1 region of the mouse hippocampus. Addition of PF4 at 1 nM, 10 nM or 100 nM in the circulating bath of mouse hippocampal slices increased field excitatory post synaptic potentials (fEPSP) in the stratum radiatum of the CA1 even in the absence of electrical stimulation, a process termed chemical synaptic plasticity.

FIG. 13. PF4 treatment increases memory in nontransgenic (NTG) young mice—in a klotho-dependent manner—following reversal of the platform location in the Morris water maze. Following daily treatment with mouse PF4 (i.p., 20 ug/kg, n=15/group, age 4 mos), mice underwent cognitive testing in the Morris watermaze. After six days of hidden training, the platform location was changed and PF4 increased learning of the new or “reversed” platform location. Two-way rpt measures ANOVA NTG: PF4 effect p<0.05; Two-tailed t-test Day 2 p=0.05 (Bonferroni-corrected).

FIG. 14A-G. Klotho induces platelet activation in the blood and increases circulating platelet factors. (14A) Paradigm for plasma proteomics profiling. Young mice (age 4 months; n=9-10 per group) were treated with either Veh or Klotho (s.c., 10 μg/kg) followed by plasma proteomics analysis. (14B) Enrichment analysis of significantly (following FDR correction) and differentially expressed proteins following Klotho treatment. (14C) Paradigm for measuring platelet activation. Young mice (age 5 months; n=8-9 mice per group) were treated with either Veh or Klotho (s.c., 10 μg/kg) followed by platelet isolation from whole blood and then platelet activation analysis by FACS sorting with markers CD61 and CD62P. (14D) Flow cytometry plots from FACS sorting showing platelet populations. The upper graphs show density plots of the platelets, gated by SSC (for granularity) and CD61-positivity. The lower graphs show dot plots of the percentage activated (CD61 and CD62P-positive) and resting (CD61-positive only) platelets. (14E) Quantification of activated platelets in young mice following treatment with Veh or Klotho. (14F) Plasma proteomics by mass spectrometry analysis 4 h after Veh or Klotho treatment identified platelet factor 4 (PF4) as the highest expressed klotho-induced protein (6.5 fold increase, FDR q-value=0.002). (14G) Quantification of mouse PF4 level by ELISA of plasma from young mice 4 hrs following treatment with Veh or Klotho. Data are presented as means±SEM; *p<0.05 (E) and (G) by two-tailed t-test.

FIG. 15A-I. Platelet factor 4 (PF4) increases synaptic plasticity through NMDAR-dependent mechanisms. (15A) Experimental paradigm of hippocampal LTP recordings from young mice genetically modified to lack mouse PF4 (PF4KO) or, in addition, overexpress human PF4 (hPF4/PBP). fEPSP recording paradigm of hPF4/PF4KO or PF4KO mice. (15B) fEPSP recordings from acute hippocampal slices of young hPF4/PBP and PF4KO mice (age 2 months; n=11-12 slices, 4 mice/group). Repeated measures ANOVA: hPF4/PBP effect, p=0.002. (15C) Average fEPSP slope over the last 10 minutes of recordings in PF4KO or hPF4/PBP. **p<0.01 by two-tailed t-test. (15D) Experimental paradigm of LTP recordings in hippocampal slices treated with either Veh or Ro 25 from PF4KO and hPF4/PBP mice. (15E) fEPSP recordings of acute hippocampal slices treated with Veh or Ro 25 from PF4KO and hPF4/PBP mice (age 3-4 months; 4-7 slices per group, n=3-4 mice per group). (15F) Average fEPSP slope over the last 10 minutes of recordings in hPF4/PBP and PF4KO mouse slices treated with either Veh or Ro 25. Two-way ANOVA: hPF4 effect, p=0.33; Ro 25 effect, p=0.21; interaction, p<0.05. *p<0.05; Bonferroni-Holm. (15G) Paradigm of Veh or mouse platelet factor 4 (mPF4) treatment (i.p., 20 μg/kg) followed by LTP induction and potentiation of the CA1 region of the hippocampus following theta burst stimulation of the Schaffer collateral pathways for fEPSP recording. Mice received daily treatment for 5-6 days. (15H) fEPSP recordings from acute hippocampal slices of young mice (age 3 months; n=4 per group, 12-13 slices per group) treated with either Veh or mPF4. *p<0.05; mPF4 effect by repeated measures ANOVA. (15I) Average fEPSP slope over the last 10 minutes of recordings in mice treated with Veh or mPF4. Data are presented as means±SEM.

FIG. 16A-K. PF4 treatment enhances cognition in young and aging mice. (16A) Diagram of the experimental paradigm of Veh or mPF4 injection (i.p. 20 μg/kg, daily) followed by testing in the elevated plus maze, open field testing, Morris water maze and the two-trial Y maze in young (age 3-5 months; n=15-18 per group) and aging (age 17-20 months mice; n=13-16 per group). (16B) Anxiety-like behavior was measured by percentage of time spent in the open arms of the elevated plus maze during 10 min exploration period of young mice treated with Veh of mPF4. (16C) Hyperactivity was measured by total activity of movements during exploration of open field for 10 min of young mice treated with Veh or mPF4. (16D) Spatial learning curves (platform hidden) of young mice treated with Veh or mPF4 in the Morris water maze. Data represent the daily average of distance travelled to find the hidden platform over two trials. Mixed model ANOVA for hidden training: mPF4 vs Vehicle, **p<0.01. (16E) Probe trial conducted 1 hr after hidden platform training and removal of the escape platform. Percentage of time the mice spent in the target quadrant of the maze, compared to the average of the other three quadrants, is shown to indicate the memory of the platform location. The dashed line represents chance performance. (16F) Spatial and working memory of young mice treated with Veh or mPF4 was assessed by the two-trial Y maze. Percentage of total distance travelled in novel and familiar arms during testing was measured 16 hours after training. (16G) Percentage of time spent in the open arms of the elevated plus maze during 10 min exploration period of aging mice treated with Veh of mPF4. (16H) Total activity of movements during exploration of open field for 10 min of aging mice treated with Veh or mPF4. (16I) Spatial learning curves (platform hidden) of aging mice treated with Veh or mPF4 in the water maze. Data represent the daily average of latency to find the hidden platform over four trials. Mixed model ANOVA for hidden training: mPF4 vs Vehicle, *p<0.05. (16J) Probe trial conducted 1 hr after hidden platform training and removal of the escape platform in aging mice. Percentage of time mice spent in the target quadrant, compared to the average of the other three quadrants, is shown to indicate memory of the platform location. The dashed line represents chance performance. (16K) Spatial and working memory of aging mice treated with Veh or mPF4 was assessed by the two-trial Y maze. Percentage of total distance travelled in novel and familiar arms during testing was measured 16 hours after training. Data are presented as means±SEM; *p<0.05, **p<0.01, ***p<0.001; Bonferroni-Holm for (C), (D), (F) and (G).

### DETAILED DESCRIPTION OF THE INVENTION

The inventors have discovered that a number of proteins that are enriched in younger blood fractions that confer cognition improvements to older mammals and/or are enriched in response to klotho, which has an established role in cognition improvement (see, e.g., U.S. Pat. No. 10,300,117). A number, but not all, of the proteins have a role in platelets.

One of the proteins, PF4 (CXCL4), has been assayed several ways and has been shown to improve cognition. Surprisingly, PF4 can be administered systemically to achieve this result, avoiding the need to directly administer the protein to the brain, for example. It is expected the other proteins identified as enriched as described herein will also be useful to enhance cognition, for example after systemic administration, in view of the result demonstrated for PF4.

Table 1 lists proteins identified as enriched in response to klotho and thus can be used as described herein, like PF4, to improve cognition as described herein. Table 2 lists proteins identified as enriched in younger blood fractions that confer cognition improvements to older mammals and thus can be used as described herein, like PF4, to improve cognition as described herein.

TABLE 1

| | | Ratio | |
|---|---|---|---|
| | | (KI/ | |
| Name | GenBank ID/Uniprot ID | Veh) | Gen. Functions |
| Platelet factor 4 | 5196 | 6.55 | PF4 is a small cytokine released from |
| (PF4, CXCL4) | (human) | | alpha-granules of activated platelets |
| | EAEEDGDLQCLCVKTTSQV | | during platelet aggregation, exercise, |
| | RPRHITSLEV IKAGPHCPTA | | and potentially other situations. It is |
| | QLIATLKNGR KICLDLQAPL | | involved in blood coagulation and |
| | YKKIIKKLLES (SEQ ID NO: 2) | | neurogenesis and is related to anti- |
| | /Q9Z126 | | cancer actions (Leiter O, Seidemann |
| | (mouse) | | S, Overall RW, et al. Stem Cell |
| | | | Reports. 2019; 12(4):667-679). Its |
| | | | receptor is CXCR (de Jong EK, de |
| | | | Haas AH, Brouwer N, et al. J |
| | | | Neurochem. 2008; 105(5):1726-1736). |
| | | | It may act more potently when |
| | | | combined with IL-8 (Nesmelova IV, |
| | | | Sham Y, Dudek AZ, et al. J Biol Chem. |
| | | | 2005; 280(6):4948-4958) (or with other |
| | | | blood factors or with klotho). |
| Thrombospondin- | 7057 | 4.04 | THB1 or TSP1 is an adhesive |
| 1 (THBS1, TSP1) | (human) | | glycoprotein involved in cell-cell and |
| | MGLAWGLGVLFLMHVCGTNRIPESGGD | | cell-matrix interactions. It plays roles in |
| | NSVFDIFELTGAARKGSGRRLVKGPD PS | | platelet aggregation, angiogenesis, |
| | SPAFRIEDANLIPPVPDDKFQDLVDAVRA | | tumorigenesis (Isenberg JS, Romeo |
| | EKGFLLLASLRQMKKTRGTLLALERKDH | | MJ, Yu C, etal. Blood. |
| | SGQVFSVVSNGKAGTLDLSLTVQGKQH | | 2008; 111(2):613-623; Sheibani N, |
| | VVSVEEALLATGQWKSITLFVQEDRAQL | | Frazier WA. Proc Natl Acad Sci USA. |
| | YIDCEKMENAELDVPIQSVFTRDLASIAR | | 1995; 92(15):6788-6792), and |
| | LRIAKGGVNDNFQGVLQNVRFVFGTTPE | | facilitates synapse formation in |
| | DILRNKGCSSSTSVLLTLDNNVVNGSSP | | hippocampal neurons through |
| | AIRTNYIGHKTKDLQAICGISCDELSSMVL | | neuroligin-1 (Xu J, Xiao N, Xia J. Nat |
| | ELRGLRTIVTTLQDSIRKVTEENKELANE | | Neurosci. 2010; 13(1):22-24). |
| | LRRPPLCYHNGVQYRNNEEWTVDSCTE | | |
| | CHCQNSVTICKKVSCPIMPCSNATVPDG | | |
| | ECCPRCWPSDSADDGWSPWSEWTSC | | |
| | STSCGNGIQQRGRSCDSLNNRCEGSSV | | |
| | QTRTCHIQECDKRFKQDGGWSHWSPW | | |
| | SSCSVTCGDGVITRIRLCNSPSPQMNGK | | |
| | PCEGEARETKACKKDACPINGGWGPWS | | |
| | PWDICSVTCGGGVQKRSRLCNNPTPQF | | |
| | GGKDCVGDVTENQICNKQDCPIDGCLS | | |
| | NPCFAGVKCTSYPDGSWKCGACPPGY | | |
| | SGNGIQCTDVDECKEVPDACFNHNGEH | | |
| | RCENTDPGYNCLPCPPRFTGSQPFGQG | | |
| | VEHATANKQVCKPRNPCTDGTHDC NKN | | |
| | AKCNYLGHYSDPMYRCECKPGYAGNGII | | |
| | CGEDTDLDGWPNENLVCVANATYHCKK | | |
| | DNCPNLPNSGQEDYDKDGIGDACDDDD | | |
| | DNDKIPDDRDNCPFHYNPAQYDYDRDD | | |
| | VGDRCDNCPYNHNPDQADTDNNGEGD | | |
| | ACAADIDGDGILNERDNCQYVYNVDQR | | |
| | DTDMDGVGDQCDNCPLEHNPDQLDSD | | |
| | SDRIGDTCDNNQDIDEDGHQNNLDNCP | | |
| | YVPNANQADHDKDGKGDACDHDDDND | | |
| | GIPDDKDNCRLVPNPDQKDSDGDGRGD | | |
| | ACKDDFDHDSVPDIDDICPENVDISETDF | | |
| | RRFQMIPLDPKGTSQNDPNWVVRHQGK | | |
| | ELVQTVNCDPGLAVGYDEFNAVDFSGT | | |
| | FFINTERDDDYAGFVFGYQSSSRFYVVM | | |
| | WKQVTQSYWDTNPTRAQGYSGLSVKV | | |
| | VNSTTGPGEHLRNALWHTGNTPGQVRT | | |
| | LWHDPRHIGWKDFTAYRWRLSHRPKTG | | |
| | FIRVVMYEGKKIMADSGPIYDKTYAGGR | | |
| | LGLFVFSQEMVFFSDLKYECRDP (SEQ | | |
| | ID NO: 3) | | |
| | /P35441 | | |
| | (mouse) | | |
| Fermitin family | 83706 | 3.95 | FERT3 is a key molecule for |
| homolog 3 | (human) | | organization of focal adhesions that |
| (FERMT3) | MAGMKTASGDYIDSSWELRVFVGEEDP | | connect cell-extracellular matrix |
| | EAESVTLRVTGESHIGGVLLKIVEQINRK | | junctions; it also controls cell-cell |
| | QDWSDHAIWWEQKRQWLLQTHWTLDK | | contacts and nucleus function (Li H, |
| | YGILADARLFFGPQHRPVILRLPNRRALR | | Deng Y, Sun K, et al. Proc Natl Acad |
| | LRASFSQPLFQAVAAICRLLSIRHPEELS | | Sci USA. 2017; 114(35):9349-9354). |
| | LLRAPEKKEKKKKEKEPEEELYDLSKVVL | | |
| | AGGVAPALFRGMPAHFSDSAQTEACYH | | |
| | MLSRPQPPPDPLLLQRLPRPSSLSDKTQ | | |
| | LHSRWLDSSRCLMQQGIKAGDALWLRF | | |
| | KYYSFFDLDPKTDPVRLTQLYEQARWDL | | |
| | LLEEIDCTEEEMMVFAALQYHINKLSQSG | | |
| | EVGEPAGTDPGLDDLDVALSNLEVKLEG | | |
| | SAPTDVLDSLTTIPELKDHLRIFRIPRRPR | | |
| | KLTLKGYRQHVWVFKETTLSYYKSQDEA | | |
| | PGDPIQQLNLKGCEVVPDVNVSGQKFCI | | |
| | KLLVPSPEGMSEIYLRCQDEQQYARWM | | |
| | AGCRLASKGRTMADSSYTSEVQAILAFL | | |
| | SLQRTGSGGPGNHPHGPDASAEGLNPY | | |
| | GLVAPRFQRKFKAKQLTPRILEAHQNVA | | |
| | QLSLAEAQLRFIQAWQSLPDFGISYVMV | | |
| | RFKGSRKDEILGIANNRLIRIDLAVGDVVK | | |
| | TWRFSNMRQWNVNWDIRQVAIEFDEHI | | |
| | NVAFSCVSASCRIVHEYIGGYIFLSTRER | | |
| | ARGEELDEDLFLQLTGGHEAF (SEQ ID | | |
| | NO: 4) | | |
| | /Q8K1B8 | | |
| | (mouse) | | |
| Talin-1 (TLN1) | 7094 | 2.77 | TLN1 is ubiquitously expressed and |
| | (human) | | mediates cell-cell adhesion by linking |
| | MVALSLKISIGNWKTMQFEPSTMVYDA | | integrins to the actin cytoskeleton; it |
| | CRIIRERIPEAPAGPPSDFGLFLSDDDPK | | also participates in the activation of |
| | KGIWLEAGKALDYYMLRNGDTMEYRKK | | integrins (Manso AM, Okada H, |
| | QRPLKIRMLDGTVKTIMVDDSKTVTDML | | Sakamoto FM, et al. Proc Natl Acad |
| | MTICARIGITNHDEYSLVRELMEEKKEEIT | | Sci USA. 2017; 114(30):E6250- |
| | GTLRKDKTLLRDEKKMEKLKQKLHTDDE | | E6259). |
| | LNWLDHGRTLREQGVEEHETLLLRRKFF | | |
| | YSDQNVDSRDPVQLNLLYVQARDDILNG | | |
| | SHPVSFDKACEFAGFQCQIQFGPHNEQ | | |
| | KHKAGFLDLKDFLPKEYVKQKGERKIFQ | | |
| | AHKNCGQMSEIEAKVRYVKLARSLKTYG | | |
| | VSFFLVKEKMKGKNKLVPRLLGITKECV | | |
| | MRVDEKTKEVIQEWNLTNIKRWAASPKS | | |
| | FTLDFGDYQDGYYSVQTTEGEQIAQLIA | | |
| | GYIDIILKKKKSKDHFGLEGDEESTMLED | | |
| | SVSPKKSTVLQQQYNRVGKVEHGSVAL | | |
| | PAIMRSGASGPENFQVGSMPPAQQQIT | | |
| | SGQMHRGHMPPLTSAQQALTGTINSSM | | |
| | QAVQAAQATLDDFDTLPPLGQDAASKA | | |
| | WRKNKMDESKHEIHSQVDAITAGTASVV | | |
| | NLTAGDPAETDYTAVGCAVTTISSNLTE | | |
| | MSRGVKLLAALLEDEGGSGRPLLQAAK | | |
| | GLAGAVSELLRSAQPASAEPRQNLLQAA | | |
| | GNVGQASGELLQQIGESDTDPHFQDAL | | |
| | MQLAKAVASAAAALVLKAKSVAQRTEDS | | |
| | GLQTQVIAAATQCALSTSQLVACTKVVA | | |
| | PTISSPVCQEQLVEAGRLVAKAVEGCVS | | |
| | ASQAATEDGQLLRGVGAAATAVTQALN | | |
| | ELLQHVKAHATGAGPAGRYDQATDTILT | | |
| | VTENIFSSMGDAGEMVRQARILAQATSD | | |
| | LVNAIKADAEGESDLENSRKLLSAAKILA | | |
| | DATAKMVEAAKGAAAHPDSEEQQQRLR | | |
| | EAAEGLRMATNAAAQNAIKKKLVQRLEH | | |
| | AAKQAAASATQTIAAAQHAASTPKASAG | | |
| | PQPLLVQSCKAVAEQIPLLVQGVRGSQA | | |
| | QPDSPSAQLALIAASQSFLQPGGKMVAA | | |
| | AKASVPTIQDQASAMQLSQCAKNLGTAL | | |
| | AELRTAAQKAQEACGPLEMDSALSVVQ | | |
| | NLEKDLQEVKAAARDGKLKPLPGETMEK | | |
| | CTQDLGNSTKAVSSAIAQLLGEVAQGNE | | |
| | NYAGIAARDVAGGLRSLAQAARGVAALT | | |
| | SDPAVQAIVLDTASDVLDKASSLIEEAKK | | |
| | AAGHPGDPESQQRLAQVAKAVTQALNR | | |
| | CVSCLPGQRDVDNALRAVGDASKRLLS | | |
| | DSLPPSTGTFQEAQSRLNEAAAGLNQA | | |
| | ATELVQASRGTPQDLARASGRFGQDFS | | |
| | TFLEAGVEMAGQAPSQEDRAQWSNLK | | |
| | GISMSSSKLLLAAKALSTDPAAPNLKSQL | | |
| | AAAARAVTDSINQLITMCTQQAPGQKEC | | |
| | DNALRELETVRELLENPVQPINDMSYFG | | |
| | CLDSVMENSKVLGEAMTGISQNAKNGN | | |
| | LPEFGDAISTASKALCGFTEAAAQAAYLV | | |
| | GVSDPNSQAGQQGLVEPTQFARANQAI | | |
| | QMACQSLGEPGCTQAQVLSAATIVAKHT | | |
| | SALCNSCRLASARTTNPTAKRQFVQSAK | | |
| | EVANSTANLVKTIKALDGAFTEENRAQC | | |
| | RAATAPLLEAVDNLSAFASNPEFSSIPAQ | | |
| | ISPEGRAAMEPIVISAKTMLESAGGLIQT | | |
| | ARALAVNPRDPPSWSVLAGHSRTVSDSI | | |
| | KKLITSMRDKAPGQLECETAIAALNSCLR | | |
| | DLDQASLAAVSQQLAPREGISQEALHTQ | | |
| | MLTAVQEISHLIEPLANAARAEASQLGHK | | |
| | VSQMAQYFEPLTLAAVGAASKTLSHPQ | | |
| | QMALLDQTKTLAESALQLLYTAKEAGGN | | |
| | PKQAAHTQEALEEAVQMMTEAVEDLTT | | |
| | TLNEAASAAGVVGGMVDSITQAINQLDE | | |
| | GPMGEPEGSFVDYQTTMVRTAKAIAVTV | | |
| | QEMVTKSNTSPEELGPLANQLTSDYGRL | | |
| | ASEAKPAAVAAENEEIGSHIKHRVQELG | | |
| | HGCAALVTKAGALQCSPSDAYTKKELIE | | |
| | CARRVSEKVSHVLAALQAGNRGTQACIT | | |
| | AASAVSGIIADLDTTIMFATAGTLNREGT | | |
| | ETFADHREGILKTAKVLVEDTKVLVQNAA | | |
| | GSQEKLAQAAQSSVATITRLADVVKLGA | | |
| | ASLGAEDPETQVVLINAVKDVAKALGDLI | | |
| | SATKAAAGKVGDDPAVWQLKNSAKVMV | | |
| | TNVTSLLKTVKAVEDEATKGTRALEATTE | | |
| | HIRQELAVFCSPEPPAKTSTPEDFIRMTK | | |
| | GITMATAKAVAAGNSCRQEDVIATANLS | | |
| | RRAIADMLRACKEAAYHPEVAPDVRLRA | | |
| | LHYGRECANGYLELLDHVLLTLQKPSPE | | |
| | LKQQLTGHSKRVAGSVTELIQAAEAMKG | | |
| | TEVWDPEDPTVIAENELLGAAAAIEAAAK | | |
| | KLEQLKPRAKPKEADESLNFEEQILEAAK | | |
| | SIAAATSALVKAASAAQRELVAQGKVGAI | | |
| | PANALDDGQWSQGLISAARMVAAATNN | | |
| | LCEAANAAVQGHASQEKLISSAKQVAAS | | |
| | TAQLLVACKVKADQDSEAMKRLQAAGN | | |
| | AVKRASDNLVKAAQKAAAFEEQENETVV | | |
| | VKEKMVGGIAQIIAAQEEMLRKERELEEA | | |
| | RKKLAQIRQQQYKFLPSELRDEH (SEQ | | |
| | ID NO: 5) | | |
| | /P26039 | | |
| | (mouse) | | |
| Creatine kinase | 1158 | 2.41 | CKM catalyzes the transfer of |
| M-type (CKM) | (human) | | phosphate between ATP and creatine; |
| | MPFGNTHNKFKLNYKPEEEYPDLSKHN | | it also catalyzes the transfer of |
| | NHMAKVLTLELYKKLRDKETPSGFTVDD | | phosphate between phospho-creatine |
| | VIQTGVDNPGHPFIMTVGCVAGDEESYE | | and ADP (Schafer B, Perriard JC, |
| | VFKELFDPIISDRHGGYKPTDKHKTDLNH | | Eppenberger HM. Basic Res Cardiol. |
| | ENLKGGDDLDPNYVLSSRVRTGRSIKGY | | 1985; 80 Suppl 2:129-133). |
| | TLPPHCSRGERRAVEKLSVEALNSLTGE | | |
| | FKGKYYPLKSMTEKEQQQLIDDHFLFDK | | |
| | PVSPLLLASGMARDWPDARGIWHNDNK | | |
| | SFLVWVNEEDHLRVISMEKGGNMKEVF | | |
| | RRFCVGLQKIEEIFKKAGHPFMWNQHLG | | |
| | YVLTCPSNLGTGLRGGVHVKLAHLSKHP | | |
| | KFEEILTRLRLQKRGTGGVDTAAVGSVF | | |
| | DVSNADRLGSSEVEQVQLVVDGVKLMV | | |
| | EMEKKLEKGQSIDDMIPAQK (SEQ ID | | |
| | NO: 6) | | |
| | /P07310 | | |
| | (mouse) | | |
| Glyceraldehyde- | 2597 | 2.05 | GAPDH is involved in catalyzing the |
| 3-phosphate | (human) | | sixth step of glycolysis; it breaks down |
| dehydrogenase | MGKVKVGVNGFGRIGRLVTRAAFNSGK | | glucose for energy and carbon |
| (GAPDH) | VDIVAINDPFIDLNYMVYMFQYDSTHGKF | | molecules (Yang JS, Hsu JW, Park |
| | HGTVKAENGKLVINGNPITIFQERDPSKI | | SY, et al. Nature. 2018; 561(7722):263- |
| | KWGDAGAEYVVESTGVFTTMEKAGAHL | | 267). |
| | QGGAKRVIISAPSADAPMFVMGVNHEKY | | |
| | DNSLKIISNASCTTNCLAPLAKVIHDNFGI | | |
| | VEGLMTTVHAITATQKTVDGPSGKLWRD | | |
| | GRGALQNIIPASTGAAKAVGKVIPELNGK | | |
| | LTGMAFRVPTANVSVVDLTCRLEKPAKY | | |
| | DDIKKWKQASEGPLKGILGYTEHQVVS | | |
| | SDFNSDTHSSTFDAGAGIALNDHFVKLIS | | |
| | WYDNEFGYSNRVVDLMAHMASKE | | |
| | (SEQ ID NO: 7) | | |
| | /P16858 | | |
| | (mouse) | | |
| Elongation factor | 1915 | 1.79 | EEF1A1 enzymatically delivers |
| 1-alpha 1 | (human) | | aminoacyl tRNAs to the ribosome |
| (EEF1A1) | MGKEKTHINIVVIGHVDSGKSTTTGHLIY | | (Vera M, Pani B, Griffiths LA, et al. |
| | KCGGIDKRTIEKFEKEAAEMGKGSFKYA | | Elife. 2014; 3:e03164). |
| | VWLDKLKAERERGITIDISLWKFETSKYY | | |
| | VTHDAPGHRDFIKNMITGTSQADCAVLIV | | |
| | AAGVGEFEAGISKNGQTREHALLAYTLG | | |
| | VKQLIVGVNKMDSTEPPYSQKRYEEIVK | | |
| | EVSTYIKKIGYNPDTVAFVPISGWNGDN | | |
| | MLEPSANMPWFKGWKVTRKDGNASGT | | |
| | TLLEALDCILPPTRPTDKPLRLPLQDVYKI | | |
| | GGIGTVPVGRVETGVLKPGMVVTFAPVN | | |
| | VTTEVKSVEMHHEALSEALPGDNVGFN | | |
| | VKNVSVKDVRRGNVAGDSKNDPPMEAA | | |
| | GFTAQVIILNHPGQISAGYAPVLDCHTAH | | |
| | IACKFAELKEKIDRRSGKKLEDGPKFLKS | | |
| | GDAAIVDMVPGKPMCVESFSDYPPLGR | | |
| | FAVRDMRQTVAVGVIKAVDKKAAGAGK | | |
| | VTKSAQKAQKAK (SEQ ID NO: 8) | | |
| | /P10126 | | |
| | (mouse) | | |
| Ig gamma-1 chain | 3500 | 1.43 | IGHG1 is a constant region of |
| C region secreted | (human) | | immunoglobulin heavy chains and is |
| form (IGHG1) | ASTKGPSVFPLAPSSKSTSGGTAALGCL | | involved in the growth of cancers (Chu |
| | VKDYFPEPVTVSWNSGALTSGVHTFPA | | J, Li Y, Deng Z, et al. IGHG1 Biomed |
| | VLQSSGLYSLSSVVTVPSSSLGTQTYICN | | Res Int. 2019; 2019:7201562). |
| | VNHKPSNTKVDKKVEPKSCDKTHTCPP | | |
| | CPAPELLGGPSVFLFPPKPKDTLMISRTP | | |
| | EVTCVVVDVSHEDPEVKFNWYVDGVEV | | |
| | HNAKTKPREEQYNSTYRVVSVLTVLHQD | | |
| | WLNGKEYKCKVSNKALPAPIEKTISKAK | | |
| | GQPREPQVYTLPPSRDELTKNQVSLTCL | | |
| | VKGFYPSDIAVEWESNGQPENNYKTTP | | |
| | PVLDSDGSFFLYSKLTVDKSRWQQGNV | | |
| | FSCSVMHEALHNHYTQKSLSLSPGK | | |
| | (SEQ ID NO: 9) | | |
| | /P01868 | | |
| | (mouse) | | |
| Tubulin alpha-1A | 7846 | 1.36 | TUBA1A is part of the formation of |
| chain (TUBA1A) | (human) | | microtubules structural proteins that |
| | MRECISIHVGQAGVQIGNACWELYCLEH | | participate in cytoskeletal structure. |
| | GIQPDGQMPSDKTIGGGDDSFNTFFSET | | Importantly, it functions in the adult |
| | GAGKHVPRAVFVDLEPTVIDEVRTGTYR | | hippocampal neurogenesis and |
| | QLFHPEQLITGKEDAANNYARGHYTIGK | | formation of dentate gyrus (Keays DA, |
| | EIIDLVLDRIRKLADQCTGLQGFLVFHSF | | Cleak J, Huang GJ, et al. Dev |
| | GGGTGSGFTSLLMERLSVDYGKKSKLE | | Neurosci. 2010; 32(4):268-277). |
| | FSIYPAPQVSTAVVEPYNSILTTHTTLEH | | |
| | SDCAFMVDNEAIYDICRRNLDIERPTYTN | | |
| | LNRLIGQIVSSITASLRFDGALNVDLTEFQ | | |
| | TNLVPYPRIHFPLATYAPVISAEKAYHEQ | | |
| | LSVAEITNACFEPANQMVKCDPRHGKY | | |
| | MACCLLYRGDVVPKDVNAAIATIKTKRTI | | |
| | QFVDWCPTGFKVGINYQPPTVVPGGDL | | |
| | AKVQRAVCMLSNTTAIAEAWARLDHKFD | | |
| | LMYAKRAFVHWYVGEGMEEGEFSEARE | | |
| | DMAALEKDYEEVGVDSVEGEGEEEGEE | | |
| | Y (SEQ ID NO: 10) | | |
| | /P68369 | | |
| | (mouse) | | |
| Heat shock | P11142(human) | 1.33 | HSPA8 facilitates proper folding of |
| cognate 71 kDa | MSKGPAVGIDLGTTYSCVGVFQHGKVEII | | newly translated and misfolded |
| protein (HSPA8) | ANDQGNRTTPSYVAFTDTERLIGDAAKN | | proteins; it also stabilizes or degrades |
| | QVAMNPTNTVFDAKRLIGRRFDDAVVQS | | mutant proteins. It fundamentally |
| | DMKHWPFMWNDAGRPKVQVEYKGET | | functions in various biological |
| | KSFYPEEVSSMVLTKMKEIAEAYLGKTV | | processes including signal |
| | TNAVVTVPAYFNDSQRQATKDAGTIAGL | | transduction, protein homeostasis, and |
| | NVLRIINEPTAAAIAYGLDKKVGAERNVLI | | cell growth/differentiation (Wang F, |
| | FDLGGGTFDVSILTIEDGIFEVKSTAGDT | | Bonam SR, Schall N, et al. Sci Rep. |
| | HLGGEDFDNRMVNHFIAEFKRKHKKDIS | | 2018; 8(1):16820). |
| | ENKRAVRRLRTACERAKRTLSSSTQASI | | |
| | EIDSLYEGIDFYTSITRARFEELNADLFRG | | |
| | TLDPVEKALRDAKLDKSQIHDIVLVGGST | | |
| | RIPKIQKLLQDFFNGKELNKSINPDEAVA | | |
| | YGAAVQAAILSGDKSENVQDLLLLDVTPL | | |
| | SLGIETAGGVMTVLIKRNTTIPTKQTQTF | | |
| | TTYSDNQPGVLIQVYEGERAMTKDNNLL | | |
| | GKFELTGIPPAPRGVPQIEVTFDIDANGIL | | |
| | NVSAVDKSTGKENKITITNDKGRLSKEDI | | |
| | ERMVQEAEKYKAEDEKQRDKVSSKNSL | | |
| | ESYAFNMKATVEDEKLQGKINDEDKQKI | | |
| | LDKCNEIINWLDKNQTAEKEEFEHQQKE | | |
| | LEKVCNPIITKLYQSAGGMPGGMPGGFP | | |
| | GGGAPPSGGASSGPTIEEVD (SEQ ID | | |
| | NO: 11) | | |
| | /P63017 (mouse) | | |
| Catalase (CAT) | 3312 | 1.27 | CAT catalyzes the decomposition of |
| | (human) | | hydrogen peroxide to water and |
| | ADSRDPASDQMQHWKEQRAAQKADVL | | oxygen (Peng J, Stevenson FF, |
| | TTGAGNPVGDKLNVITVGPRGPLLVQDV | | Doctrow SR, Andersen JK. J Biol |
| | VFTDEMAHFDRERIPERVVHAKGAGAF | | Chem. 2005; 280(32):29194-29198) |
| | GYFEVTHDITKYSKAKVFEHIGKKTPIAV | | |
| | RFSTVAGESGSADTVRDPRGFAVKFYT | | |
| | EDGNWDLVGNNTPIFFIRDPILFPSFIHS | | |
| | QKRNPQTHLKDPDMVWDFWSLRPESL | | |
| | HQVSFLFSDRGIPDGHRHMNGYGSHTF | | |
| | KLVNANGEAVYCKFHYKTDQGIKNLSVE | | |
| | DAARLSQEDPDYGIRDLFNAIATGKYPS | | |
| | WTFYIQVMTFNQAETFPFNPFDLTKVWP | | |
| | HKDYPLIPVGKLVLNRNPVNYFAEVEQIA | | |
| | FDPSNMPPGIEASPDKMLQGRLFAYPDT | | |
| | HRHRLGPNYLHIPVNCPYRARVANYQR | | |
| | DGPMCMQDNQGGAPNYYPNSFGAPEQ | | |
| | QPSALEHSIQYSGEVRRFNTANDDNVTQ | | |
| | VRAFYVNVLNEEQRKRLCENIAGHLKDA | | |
| | QIFIQKKAVKNFTEVHPDYGSHIQALLDK | | |
| | YNAEKPKNAIHTFVQSGSHLAAREKANL | | |
| | (SEQ ID NO: 12) | | |
| | /P24270 | | |
| | (mouse) | | |
| Actin, cytoplasmic | 60; 71 | 1.23 | ACTG1 polymerizes to make filaments |
| 1; Actin, | (human) | | by forming cross-linked networks in the |
| cytoplasmic 2 | MDDDIAALVVDNGSGMCKAGFAGDDAP | | cytoplasm of cells to facilitate motility |
| (ACTB; ACTG1) | RAVFPSIVGRPRHQGVMVGMGQKDSYV | | and contraction (Hsueh YP. Commun |
| | GDEAQSKRGILTLKYPIEHGIVTNWDDM | | Integr Biol. 2012; 5(4):334-336.) |
| | EKIWHHTFYNELRVAPEEHPVLLTEAPL | | |
| | NPKANREKMTQIMFETFNTPAMYVAIQA | | |
| | VLSLYASGRTTGIVMDSGDGVTHTVPIY | | |
| | EGYALPHAILRLDLAGRDLTDYLMKILTE | | |
| | RGYSFTTTAEREIVRDIKEKLCYVALDFE | | |
| | QEMATAASSSSLEKSYELPDGQVITIGN | | |
| | ERFRCPEALFQPSFLGMESCGIHETTFN | | |
| | SIMKCDVDIRKDLYANTVLSGGTTMYPGI | | |
| | ADRMQKEITALAPSTMKIKIIAPPERKYSV | | |
| | WIGGSILASLSTFQQMWISKQEYDESGP | | |
| | SIVHRKCF (SEQ ID NO: 13) | | |
| | /P60710; P63260 | | |
| | (mouse) | | |
| Hemoglobin | 15129 | 1.21 | HBB-B1 is the most common form of |
| subunit beta-1 | (mouse) | | hemoglobin in adult humans; it is |
| (HBB-B1) | MVHLTPEEKSAVTALWGKVNVDEVGGE | | involved in some genetic disorders |
| | ALGRLLVVYPWTQRFFESFGDLSTPDAV | | such as sickle-cell and beta |
| | MGNPKVKAHGKKVLGAFSDGLAHLDNL | | thalassemia (Chang CK, Simplaceanu |
| | KGTFATLSELHCDKLHVDPENFRLLGNV | | V, Ho C. Biochemistry. |
| | LVCVLAHHFGKEFTPPVQAAYQKVVAGV | | 2002; 41(17):5644-5655). |
| | ANALAHKYH (SEQ ID NO: 14) | | |
| | /P02088 | | |
| | (mouse) | | |
| Phospholipid | 5360 | 1.17 | PLTP transfers phospholipids from |
| transfer protein | (human) | | triglyceride-rich lipoproteins to high |
| (PLTP) | MALFGALFLALLAGAHAEFPGCKIRVTSK | | density lipoprotein (HDL) and is |
| | ALELVKQEGLRFLEQELETITIPDLRGKE | | involved in cholesterol metabolism |
| | GHFYYNISEVKVTELQLTSSELDFQPQQ | | (DesrumauxC, Risold PY, Schroeder |
| | ELMLQITNASLGLRFRRQLLYWFFYDGG | | H, etal. FASEB J. 2005; 19(2):296- |
| | YINASAEGVSIRTGLELSRDPAGRMKVS | | 297). |
| | NVSCQASVSRMHAAFGGTFKKVYDFLS | | |
| | TFITSGMRFLLNQQICPVLYHAGTVLLNS | | |
| | LLDTVPVRSSVDELVGIDYSLMKD PVAS | | |
| | TSNLDMDFRGAFFPLTERNWSLPNRAV | | |
| | EPQLQEEERMVYVAFSEFFFDSAMESY | | |
| | FRAGALQLLLVGDKVPHDLDMLLRATYF | | |
| | GSIVLLSPAVIDSPLKLELRVLAPPRCTIK | | |
| | PSGTTISVTASVTIALVPPDQPEVQLSSM | | |
| | TMDARLSAKMALRGKALRTQLDLRRFRI | | |
| | YSNHSALESLALIPLQAPLKTMLQIGVMP | | |
| | MLNERTWRGVQIPLPEGINFVHEVVTNH | | |
| | AGFLTIGADLHFAKGLREVIEKNRPADVR | | |
| | ASTAPTPSTAAV (SEQ ID NO: 15) | | |
| | /P55065 | | |
| | (mouse) | | |
| Complement C4- | 721 | 1.13 | C4B participates in the complement |
| B (C4B) | (human) | | system, is derived from human |
| | MRLLWGLIWASSFFTLSLQKPRLLLFSPS | | leukocyte antigen (HLA), and functions |
| | VVHLGVPLSVGVQLQDVPRGQVVKGSV | | in immunity (Agarwal V, Talens S, |
| | FLRNPSRNNVPCSPKVDFTLSSERDFAL | | Grandits AM, Blom AM. J Biol Chem. |
| | LSLQVPLKDAKSCGLHQLLRGPEVQLVA | | 2015; 290(30): 18333-18342). |
| | HSPWLKDSLSRTTNIQGINLLFSSRRGHL | | |
| | FLQTDQPIYNPGQRVRYRVFALDQKMR | | |
| | PSTDTITVMVENSHGLRVRKKEVYMPSS | | |
| | IFQDDFVIPDISEPGTWKISARFSDGLES | | |
| | NSSTQFEVKKYVLPNFEVKITPGKPYILT | | |
| | VPGHLDEMQLDIQARYIYGKPVQGVAYV | | |
| | RFGLLDEDGKKTFFRGLESQTKLVNGQS | | |
| | HISLSKAEFQDALEKLNMGITDLQGLRLY | | |
| | VAAAIIESPGGEMEEAELTSWYFVSSPF | | |
| | SLDLSKTKRHLVPGAPFLLQALVREMSG | | |
| | SPASGIPVKVSATVSSPGSVPEVQDIQQ | | |
| | NTDGSGQVSIPIIIPQTISELQLSVSAGSP | | |
| | HPAIARLTVAAPPSGGPGFLSIERPDSRP | | |
| | PRVGDTLNLNLRAVGSGATFSHYYYMIL | | |
| | SRGQIVFMNREPKRTLTSVSVFVDHHLA | | |
| | PSFYFVAFYYHGDHPVANSLRVDVQAG | | |
| | ACEGKLELSVDGAKQYRNGESVKLHLET | | |
| | DSLALVALGALDTALYAAGSKSHKPLNM | | |
| | GKVFEAMNSYDLGCGPGGGDSALQVF | | |
| | QAAGLAFSDGDQWTLSRKRLSCPKEKT | | |
| | TRKKRNVNFQKAINEKLGQYASPTAKRC | | |
| | CQDGVTRLPMMRSCEQRAARVQQPDC | | |
| | REPFLSCCQFAESLRKKSRDKGQAGLQ | | |
| | RALEILQEEDLIDEDDIPVRSFFPENWLW | | |
| | RVETVDRFQILTLWLPDSLTTWEIHGLSL | | |
| | SKTKGLCVATPVQLRVFREFHLHLRLPM | | |
| | SVRRFEQLELRPVLYNYLDKNLTVSVHV | | |
| | SPVEGLCLAGGGGLAQQVLVPAGSARP | | |
| | VAFSVVPTAATAVSLKVVARGSFEFPVG | | |
| | DAVSKVLQIEKEGAIHREELVYELNPLDH | | |
| | RGRTLEIPGNSDPNMIPDGDFNSYVRVT | | |
| | ASDPLDTLGSEGALSPGGVASLLRLPRG | | |
| | CGEQTMIYLAPTLAASRYLDKTEQWSTL | | |
| | PPETKDHAVDLIQKGYMRIQQFRKADGS | | |
| | YAAWLSRGSSTWLTAFVLKVLSLAQEQV | | |
| | GGSPEKLQETSNWLLSQQQADGSFQDL | | |
| | SPVIHRSMQGGLVGNDETVALTAFVTIAL | | |
| | HHGLAVFQDEGAEPLKQRVEASISKASS | | |
| | FLGEKASAGLLGAHAAAITAYALTLTKAP | | |
| | ADLRGVAHNNLMAMAQETGDNLYWGS | | |
| | VTGSQSNAVSPTPAPRNPSDPMPQAPA | | |
| | LWIETTAYALLHLLLHEGKAEMADQAAA | | |
| | WLTRQGSFQGGFRSTQDTVIALDALSAY | | |
| | WIASHTTEERGLNVTLSSTGRNGFKSHA | | |
| | LQLNNRQIRGLEEELQFSLGSKINVKVG | | |
| | GNSKGTLKVLRTYNVLDMKNTTCQDLQI | | |
| | EVTVKGHVEYTMEANEDYEDYEYDELP | | |
| | AKDDPDAPLQPVTPLQLFEGRRNRRRR | | |
| | EAPKVVEEQESRVHYTVCIWRNGKVGL | | |
| | SGMAIADVTLLSGFHALRADLEKLTSLSD | | |
| | RYVSHFETEGPHVLLYFDSVPTSRECVG | | |
| | FEAVQEVPVGLVQPASATLYDYYNPERR | | |
| | CSVFYGAPSKSRLLATLCSAEVCQCAEG | | |
| | KCPRQRRALERGLQDEDGYRMKFACYY | | |
| | PRVEYGFQVKVLREDSRAAFRLFETKIT | | |
| | QVLHFTKDVKAAANQMRNFLVRASCRL | | |
| | RLEPGKEYLIMGLDGATYDLEGHPQYLL | | |
| | DSNSWIEEMPSERLCRSTRQRAACAQL | | |
| | NDFLQEYGTQGCQV (SEQ ID NO: 16) | | |
| | /P01029 | | |
| | (mouse) | | |
| Beta-enolase | 2027 | 1.09 | ENO3 is found in skeletal muscle cells |
| (ENO3) | (human) | | and could play a role in muscle |
| | MAMQKIFAREILDSRGNPTVEVDLHTAK | | development and regeneration |
| | GRFRAAVPSGASTGIYEALELRDGDKGR | | (Peshavaria M, Day IN. Biochem J. |
| | YLGKGVLKAVENINNTLGPALLQKKLSVV | | 1993; 292 (Pt 3):701-704). |
| | DQEKVDKFMIELDGTENKSKFGANAILG | | |
| | VSLAVCKAGAAEKGVPLYRHIADLAGNP | | |
| | DLILPVPAFNVINGGSHAGNKLAMQEFMI | | |
| | LPVGASSFKEAMRIGAEVYHHLKGVIKA | | |
| | KYGKDATNVGDEGGFAPNILENNEALEL | | |
| | LKTAIQAAGYPDKVVIGMDVAASEFYRN | | |
| | GKYDLDFKSPDDPARHITGEKLGELYKS | | |
| | FIKNYPVVSIEDPFDQDDWATWTSFLSG | | |
| | VNIQIVGDDLTVTNPKRIAQAVEKKACNC | | |
| | LLLKVNQIGSVTESIQACKLAQSNGWGV | | |
| | MVSHRSGETEDTFIADLVVGLCTGQIKT | | |
| | GAPCRSERLAKYNQLMRIEEALGDKAIF | | |
| | AGRKFRNPKAK (SEQ ID NO: 17) | | |
| | /P21550 | | |
| | (mouse) | | |
| Ig heavy chain V | /P06330 | 1.08 | Unknown |
| region AC38 | (mouse) | | |
| 205.12 (NAN) | EVQLQQSGPELVKPGASVKISCKASGYT | | |
| | FTDYYMNWVKQSHGKSLEWIGDINPNN | | |
| | GGTSYNQKFKGKATLTVDKSSSATYMEL | | |
| | RSLTSEDSAVYYCARGYGYDPFDVWGT | | |
| | GTTVTVSS (SEQ ID NO: 18) | | |

**All listed proteins were significantly induced by klotho treatment in the plasma of mice following correction for multiple testing.

TABLE 2

| Name | GenBank ID/Uniprot ID | Gen. Functions |
|---|---|---|
| Platelet factor 4 (PF4, | 5196 (human) | PF4 is a small cytokine released from |
| CXCL4) | (SEQ ID NO: 1)/ | alpha-granules of activated platelets during |
| | Q9Z126 (mouse) | platelet aggregation, exercise, and |
| | | potentially other situations. It is involved in |
| | | blood coagulation, immunomodulating, and |
| | | neurogenesis and is related to anti-cancer |
| | | actions (Leiter, O. et al. Stem cell reports |
| | | 12, 667-679 (2019)). Its receptor is CXCR3 |
| | | (de Jong, E. K. et al. J. Neurochem. 105, |
| | | 1726-1736 (2008)). It may act more |
| | | potently when combined with IL-8 |
| | | (Nesmelova, I. V et al. J. Biol. Chem. 280, |
| | | 4948-58 (2005)). |
| SH3 domain-binding | 83442 (human) | SH3BGRL3 or TIPB1 belongs to the |
| glutamic acid-rich-like | Msglrvystsvtgsreiksqqsevtrildg | thioredoxin-like protein family, but it lacks the |
| protein 3 (SH3BGRL3 | kriqyqlvdisqdnalrdemralagnpk | CxxC motif essential for catalytic activity |
| or TIPB1) | atppqivngdqycgdyelfveaveqntl | (Mazzocco, M. etal. Biochem. Biophys. Res. |
| | qeflkla (SEQ ID NO: 20)/ | Commun. 285, 540-545 (2001).). However, |
| | Q91VW3 (mouse) | it has been shown to protect several cell |
| | | lines from lysis induced by high doses of |
| | | TNFα ( Berleth, E. S. et al. Cancer Res. 59, |
| | | 5497-506 (1999)). |
| Actin-related protein | 10094 (human) | The Arp2/3 protein complex has been |
| 2/3 complex subunit 3 | Mpayhsslmdpdtklignmallpirsqf | implicated in the control of actin |
| (ARPC3) | kgpapretkdtdivdeaiyyfkanvffkn | polymerization in cells (Goley, E. D. & |
| | yeikneadrtliyitlyiseclkklqkcnsks | Welch, M. D. Nature Reviews Molecular Cell |
| | qgekemytlgitnfpipgepgfplnaiya | Biology 7, 713-726 (2006)). |
| | kpankqedevmraylqqlrqetglrlce | |
| | kvfdpqndkpskwwtcfvkrqfmnksl | |
| | sgpgq (SEQ ID NO: 21)/ | |
| | Q9JM76 (mouse) | |
| CD151 molecule | 977 (human) | CD151 or PETA-3 is a cell surface |
| (Raph blood group); | Mgefnekkttcgtvclkyllftynccfwla | glycoprotein that is known to complex with |
| (CD151 or PETA-3) | glavmavgiwtlalksdyisllasgtylata | integrins and other transmembrane 4 |
| | yilvvagtvvmvtgvigccatfkerrnllrly | superfamily proteins. It is involved in cell |
| | fillliiflleiiagilayayyqqlntelkenlkdt | adhesion and may regulate integrin |
| | mtkryhqpgheavtsavdqlqqefhcc | trafficking and/or function. It mediates signal |
| | gsnnsqdwrdsewirsqeaggrvvpd | transduction events that play a role in the |
| | sccktvvalcgqrdhasniykveggcitk | regulation of cell development, activation, |
| | letfiqehlrvigavgigiacvqvfgmiftcc | growth and motility (Sincock, P. M. et al. J. |
| | lyrsiklehy (SEQ ID NO: 22)/ | Cell Sci. 112, 833-844 (1999)). |
| | O35566 (mouse) | |
| F2R like thrombin or | 9002 (human) | F2RL3 or PAR4 is a member of the |
| trypsin receptor 3 | Mwgrlllwplvlgfslsggtqtpsvydes | protease-activated receptor subfamily, which |
| (F2RL3 or PAR4) | gstgggddstpsilpaprgypgqvcan | is proteolytically processed to reveal an |
| | dsdtlelpdssralllgwvptrlvpalyglvl | extracellular N-terminal tethered ligand that |
| | wglpanglalwvlatqaprlpstmllmnl | binds to and activates the receptor (Xu, W. |
| | aaadlllalalppriayhlrgqrwpfgeaa | F. et al. Proc. Natl. Acad. Sci. U.S.A. 95, |
| | crlataalyghmygsvlllaavsldrylalv | 6642-6646 (1998)). It plays a role in platelet |
| | hplraralrgrrlalglcmaawlmaaalal | activation, inflammation, and response to |
| | pltlqrqtfrlarsdrvlchdalpldaqash | pain (Kahn, M. L., Nakanishi-Matsui, M., |
| | wqpaftclallgcflpllamllcygatlhtla | Shapiro, M. J., Ishihara, H. & Coughlin, S. R. |
| | asgrryghalrltavvlasavaffvpsnlllll | J. Clin. Invest. 103, 879-887 (1999); Wang, |
| | hysdpspsawgnlygayvpslalstlns | Z. etai. J. Neurosci. Res. 91, 1551-1562 |
| | cvdpfiyyyvsaefrdkvraglfqrspgdt | (2013)). |
| | vaskasaeggsrgmgthssllq | |
| | (SEQ ID NO: 23)/ | |
| | O88634 (mouse) | |
| C-type lectin domain | 51266 (human) | CLEB2B or CLEC-2 is highly expressed on |
| family 1 member B | Mqdedgyitlniktrkpalisvgsasssw | platelets and megakaryocytes. Deletion from |
| (CLEC1B or CLEC-2) | wrvmalillilcvgmvvglvalgiwsvmq | platelets leads to impairments in hemostasis |
| | mylqgenenrtgtlqqlakrfcqyvvkqs | (Bender, M. et al. Arterioscler. Thromb. |
| | elkgtfkghkcspcdtnwryygdscygff | Vase. Biol. 33, 926-934 (2013)) and |
| | rhnltweeskqyctdmnatllkidnrniv | enhanced systemic inflammation in models |
| | eyikarthlirwvglsrqksnevwkwed | of sepsis (Rayes, J. et al. Nat. Commun. 8, |
| | gsvisenmfefledgkgnmncayfhng | 1-14 (2017)). |
| | kmhptfcenkhylmcerkagmtkvdqlp | |
| | (SEQ ID NO: 24)/ | |
| | Q9JL99 (mouse) | |
| Linker For Activation | 27040 (human) | Upon activation, LAT forms numerous |
| Of T Cells (LAT) | Meeailvpcvlgllllpilamlmalcvhch | interactions with other signaling molecules, |
| | rlpgsydstssdslyprgiqfkrphtvap | leading to enhanced intracellular signaling, |
| | wppayppvtsypplsqpdllpiprspqp | most notably following activation of the T cell |
| | lggshrtpssrrdsdgansvasyeneg | antigen receptor signal transduction pathway |
| | asgirgaqagwgvwgpswtrltpvslp | (Hor̆ejs̆í, V. Transmembrane adaptor |
| | pepacedadededdyhnpgylvvlpd | proteins in membrane microdomains: |
| | stpatstaapsapalstpgirdsafsmes | Important regulators of immunoreceptor |
| | iddyvnvpesgesaeasldgsreyvnv | signaling, in Immunology Letters 92, 43-49 |
| | sqelhpgaaktepaalssqeaeevee | (Immunol Lett, 2004)). LAT is also expressed |
| | egapdyenlqeln | in platelets and plays opposing roles in |
| | (SEQ ID NO: 25)/ | aggregation induced by collagen, and |
| | O54957 (mouse) | thromboxane A2 or ADP (Cho, M. J., |
| | | Gartner, T. K., Pestina, T. I., Steward, S. A. |
| | | & Jackson, C. W. Biochem. Biophys. Res. |
| | | Commun. 292, 916-921 (2002)). |
| Tubulin beta 1 chain | 81027 (human) | Tubulin is the major constituent of |
| (TUBB1) | Mreivhiqigqcgnqigakfwemigee | microtubules. Megakaryocytes and platelets |
| | hgidlagsdrgasalqlerisvyyneayg | primarily express the TUBB1 isotype. |
| | rkyvpravlvdlepgtmdsirssklgalfq | Microtubules in platelets provide structural |
| | pdsfvhgnsgagnnwakghytegaeli | integrity. TUBB1 null mice have moderate |
| | envlevvrhesescdclqgfqivhslgg | thrombocytopenia and prolonged mean tail |
| | gtgsgmgtllmnkireeypdrimnsfsv | bleeding times (Burley, K., Westbury, S. K. & |
| | mpspkvsdtvvepynavlsihqlienad | Mumford, A. D., Platelets 29, 209-211 |
| | acfcidnealydicfrtlklttptygdlnhlvs | (2018)). |
| | ltmsgittslrfpgqlnadlrklavnmvpfp | |
| | rlhffmpgfapltaqgsqqyralsvaeltq | |
| | qmfdamtmaacdlrrgryltvacifrgk | |
| | mstkevdqqllsvqtrnsscfvewipnn | |
| | vkvavcdipprglsmaatfignntaiqeif | |
| | nrvsehfsamfkrkafvhwytsegmdi | |
| | nefgeaennihdlvseyqqfqdakavle | |
| | edeevteeaemepedkgh | |
| | (SEQ ID NO: 26)/ | |
| | A2AQ07 (mouse) | |
| F-actin-capping | 829 (human) | CAPZA1 regulates growth of the actin |
| protein subunit alpha-1 | Madfddrvsdeekvriaakfithappge | filament by capping the barbed end (plus- |
| (CAPZA1) | fnevfndvrlllnndnllregaahafaqyn | end) of growing actin filaments, preventing |
| | mdqftpvkiegyedqvlitehgdlgnsrfl | any further assembly from occurring. Binding |
| | dprnkisfkfdhlrkeasdpqpeeadgg | to PIP2 can prevent CAPZA1 from binding to |
| | Ikswrescdsalrayvkdhysngfctvy | actin filaments (Maun, N. A., Speicher, D. |
| | aktidgqqtiiacieshqfqpknfwngrw | W., DiNubile, M. J. & Southwick, F. S. |
| | rsewkftitpptaqvvgvlkiqvhyyedg | Biochemistry 35, 3518-3524 (1996)). |
| | nvqlvshkdvqdsltvsneaqtakefikii | |
| | enaeneyqtaisenyqtmsdttfkalrrq | |
| | Ipvtrtkidwnkilsykigkemqna | |
| | (SEQ ID NO: 27)/ | |
| | P47753 (mouse) | |
| S-formylglutathione | 2098 (human) | FGH or ESD is an enzyme that functions as |
| hydrolase (FGH or | Malkqissnkcfgglqkvfehdsvelnc | a serine hydrolase, which is involved in the |
| ESD) | kmkfavylppkaetgkcpalywlsgltct | detoxification of formaldehyde (Young, L. J. |
| | eqnfisksgyhqsasehglvviapdtsp | et al. Hum. Genet. 79, 137-41 (1988)). |
| | rgcnikgedeswdfgtgagfyvdated | |
| | pwktnyrmysyvteelpqlinanfpvdp | |
| | qrmsifghsmgghgalicalknpgkyk | |
| | svsafapicnpvlcpwgkkafsgylgtd | |
| | qskwkaydathlvksypgsqldilidqg | |
| | kddqflldgqllpdnfiaactekkipwfrl | |
| | qegydhsyyfiatfitdhirhhakylna | |
| | (SEQ ID NO: 28)/ | |
| | Q9ROP3 (mouse) | |

**Top 10 proteins elevated in the platelet-enriched fraction of plasma from young mice (2 months) relative to old mice (20 months).

As noted above, in some embodiments, the polypeptide administered is PF4, or a functional fragment or variant thereof. In some embodiments, the polypeptide administered is at least 70, 75, 80, 85, 90, 95, 97, or 99% identical to SEQ ID NO:1 or SEQ ID NO:19.

In some embodiments, PF4 is modified with one or more amino acid changes, relative to SEQ ID NO:1 or 19, to reduce or avoid heparin induced thrombocytopenia (HIT). HIT is caused by antibodies that bind to complexes of heparin and PF4, activating the platelets and promoting a prothrombotic state. See, e.g., Cai et al., Nature Communications volume 6, Article number: 8277 (2015). In some embodiments, the PF4 includes an amino acid change relative to SEQ ID NO:1 of one or more of the following positions: C10, C12, C36, C52, 1142, which can be changed to alanine or another amino acid. See, e.g., Huynh, et al., J Thrombosis and Haemostasis 17: 389-399 24 Dec. 2018.

PF-4 and Interleukin-8 (IL-8) can form heterodimers. See, e.g., Nesmelova, et al., J. Biol. Chem, 280, 4948-4958 (2005). In some embodiments in which PF4 or a functional fragment or variant thereof is administered to a subject, IL-8 is also administered to the subject. Administration of PF4 and IL-8 can be simultaneous or sequential. In some embodiments, PF4 is administered within (e.g., before, after or both) 1, 2, 4, 8, 12, 24, or 48 hours of administration of IL-8. In some embodiments, PF4 or a functional fragment or variant thereof is formulated with IL-8 in a single pharmaceutical composition that can be administered to the subject.

In some embodiments, the polypeptide is at least 70, 75, 80, 85, 90, 95, 97, or 99% identical to a protein set forth in Table 1 or Table 2 (e.g., any one of SEQ ID NO: 2-18 or 20-28).

In some embodiments, instead of administering a polypeptide as set forth in Table 1 or Table 2, an agonist of a receptor of a polypeptide of Table 1 or Table 2 can be administered to achieve the same effect. For example, PF4 is an agonist of receptors CXCR3 and CCR1. Accordingly, in some embodiments, an agonist of CXCR3 and CCR1 is administered as described herein to improve cognition. Examples of CXCR3 agonists are described in, e.g., WO2018045246; Stroke et al “Identification of CXCR3 receptor agonists in combinatorial small-molecule libraries,” Biochemical and Biophysical Research Communication, 349:221-228, 2006; and O'Boyle et al “Chemokine receptor CXCR3 agonist prevents human T-cell migration in a humanized model of arthritic inflammation,” PNAS, 109(12):4598-4603, 2012. Additional CXCR3 agonists include CXCL9, CXCL10, and CXCL11. See, e.g., Colvin, et al, J Biol Chem. 2004 Jul. 16; 279(29):30219-27. CCR1 agonists are described in, e.g., Lee, et al., J Leukoc Biol. 2009 December; 86(6):1319-29. Additional CCR1 agonists include CCL2, CCL3, CCL7, and CCL8. See, e.g., Azizi et al, Am J Alzheimers Dis Other Demen 2014 Aug. 29 (5), 415-25.

Thrombospondin-1 (THBS1, TSP1) binds CD47. See, e.g., Resovi et al, Matrix Biol. 2014 July; 37:83-91. PKHB1 is an agonist for CD47 and induces immunogenic functions, but has not been described for cognitive functions. See, e.g., Uscanga-Palomeque et al, Cancer Sci. 2019 January; 110(1): 256-268. In some embodiments of the methods described herein, cognition is improved in a subject in need thereof by administering to the subject an effective amount of PKHB1 or a function fragment or variant thereof.

Polypeptides that can be used for administration include species homologs (e.g., non-human primate, mouse, rat), allelic variants (human or other), functional fragments, and functional variants of the wild type sequence of any of the polypeptides in Table 1 or Table 2 that retain cognition-improving activity. Examples include variants comprising at least one (e.g., 1, 2, 3, 4, 5, 6, 7, 8, 9, 10, e.g., 1-20, 1-5) conserved or non-conserved amino acid in the naturally-occurring protein substituted with a different amino acid or deleted. In some embodiments, the polypeptide is at least 70, 75, 80, 85, 90, 95, 97, or 99% identical to a polypeptide as set forth in Table 1 or Table 2. In some embodiments, a functional fragment comprises at least 40, 50, 60, 70 or more contiguous amino acids of a naturally-occurring polypeptide of Table 1 or Table 2. For example, in some embodiments, the functional fragment comprises the naturally-occurring polypeptide sequence but lacks 1, 2, 3, 4, 5, 6, 7, 8, 9, 10 or more amino acids at the amino terminus of the naturally-occurring polypeptide. In some embodiments, the functional fragment comprises the naturally-occurring polypeptide sequence but lacks 1, 2, 3, 4, 5, 6, 7, 8, 9, 10 or more amino acids at the carboxyl terminus of the naturally-occurring polypeptide. The functional fragment can in some embodiments be part of a fusion protein linked to a heterologous amino acid sequence.

In some embodiments, the polypeptide is part of a larger fusion protein. In some embodiments, the fusion protein comprises a polypeptide as described herein in Table 1 or

Table 2 and further comprises no more than 100, 75, 50, or 30 additional amino acids. In some embodiments, the polypeptide comprises (e.g., is fused to) an affinity tag (e.g., a histidine tag) or a conjugate to increase stability or half-life in vivo. In some embodiments, the polypeptide is PEGylated to increase stability or half-life in vivo.

A functional variant or fragment of a polypeptide described herein is a variant or fragment that retains a measurable (e.g., cognition-improving) activity, e.g., at least 20%, 30%, 40%, 50%, 60%, 70%, 80%, 90%, or 100% of the level of the naturally-occurring polypeptide. Activity can be measured by, e.g., causing changes in magnetic resonance imaging (MRI) brain scans, e.g., functional MRI, electroencephalograph (EEG), and transcranial magnetic and electrical stimulation (TMS and TES); and improved performance in neuropsychologic testing and cognitive ability.

Provided herein are methods of improving cognition and/or motor function in an individual comprising administering a polypeptide as described herein (e.g., as listed in Table 1 or Table 2) to the individual. In some embodiments, the method of treatment comprises administering to an individual an effective amount of the polypeptide (or functional variant or fragment thereof). In some embodiments, the treatment is prophylactic, e.g., for an individual expecting stress (e.g., jet lag, military performance) or to prevent cognitive decline associated with aging. In some embodiments, the individual has been diagnosed with a cognitive disorder. In some embodiments, the individual is receiving or has received therapy for a cognitive disorder or for a condition that is related to cognitive function (e.g., cognitive decline in response to chemotherapy).

In some embodiments, the method further comprises monitoring the individual for cognitive ability, either through a molecular proxy (e.g., changes NMDA receptor or c-fos activation, or GluN2B levels in the brain), changes in MRI brain scans (e.g., functional MRI), changes in EEG, changes in TMS and TES, changes in neuropsychologic test scores, or tests of cognitive ability (e.g., for learning, short or long term memory, executive functions, language ability, and visuospatial function). In some embodiments, the individual is monitored using more than one of the above tests in any combination. In some embodiments, the dose of the polypeptide for each administration is determined based on the therapeutic progress of the individual, e.g., where a higher dose is administered if the individual is not responding sufficiently to therapy.

In some embodiments, the polypeptide is administered in a pharmaceutical composition with a physiologically (i.e., pharmaceutically) acceptable carrier. The term “carrier” refers to a typically inert substance used as a diluent or vehicle for a diagnostic or therapeutic agent. The term also encompasses a typically inert substance that imparts cohesive qualities to the composition. Physiologically acceptable carriers can be liquid, e.g., physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17th ed., 1989).

The presently described compositions can be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized antibody compositions.

Dosage forms can be prepared for mucosal (e.g., nasal, sublingual, vaginal, buccal, or rectal), parenteral (e.g., subcutaneous, intravenous, intramuscular, or intraarterial injection, either bolus or infusion), oral, or transdermal administration to a patient. Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Injectable compositions can comprise a solution of the polypeptide suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. In some embodiments, normal buffered saline (135-150 mM NaCl) is used. The compositions can contain pharmaceutically acceptable auxiliary substances to approximate physiological conditions, such as pH adjusting and buffering agents, tonicity adjusting agents, wetting agents, e.g., sodium acetate, sodium lactate, sodium chloride, potassium chloride, calcium chloride, sorbitan monolaurate, triethanolamine oleate, etc.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In some embodiments, the composition is administered by intravenous infusion, topically, intraperitoneally, intravesically, or intrathecally. The polypeptide formulation can be provided in unit-dose or multi-dose sealed containers, such as ampoules and vials.

The polypeptide composition, alone or in combination with other suitable components, can be made into aerosol formulations (“nebulized”) to be administered via inhalation. Aerosol formulations can be placed into pressurized acceptable propellants, such as dichlorodifluoromethane, propane, and nitrogen.

The pharmaceutical preparation can be packaged or prepared in unit dosage form. In such form, the preparation is subdivided into unit doses containing appropriate quantities of the active component, e.g., according to the dose of polypeptide. The unit dosage form can be a packaged preparation, the package containing discrete quantities of preparation. The composition can, if desired, also contain other compatible therapeutic agents. In some embodiments, the polypeptide composition can be formulated in a kit for administration.

In some embodiments, a pharmaceutical composition comprising a polypeptide as described herein is administered orally. In some embodiments, a pharmaceutical composition comprising a polypeptide is administered mucosally, e.g., nasally. In some embodiments, a pharmaceutical composition comprising a polypeptide is administered by injection, e.g., subcutaneous, intraperitoneal, intravenous, or intramuscular. In some embodiments, a pharmaceutical composition comprising a polypeptide is administered by infusion, e.g., using a reservoir or osmotic minipump.

An example of administration of a pharmaceutical composition includes storing the polypeptide at 10 mg/ml in sterile isotonic aqueous saline solution at 4° C., and diluting it in an appropriate solution for injection prior to administration to the patient. In some embodiments, the polypeptide composition can be administered by intravenous infusion over the course of 0.25-2 hours. In some embodiments, the administration procedure is via bolus injection.

In some embodiments, in therapeutic use, the polypeptide can be administered at the initial dosage of about 0.1 μg/kg to about 1000 μg/kg daily and adjusted over time. For example, in some embodiments, a daily dose range of about 1 μg/kg to about 500 μg/kg, or about 10 μg/kg to about 100 μg/kg, or about 30 μg/kg to about 50 μg/kg can be used. The dosage is varied depending upon the requirements of the patient, the severity of the condition being treated, and the route of administration. For example, in some embodiments, for injection of the polypeptide, the effective dose can typically in the range of 10-100 μg/kg, while for direct delivery to the central nervous system (CNS), the effective dosage is lower, e.g., 5-30 μg/kg. For oral administration, in some embodiments, the effective dose is higher, e.g., in the range of 50-10,000 μg/kg (e.g., 100 μg/kg-2 mg/kg). The dose is chosen in order to provide effective therapy for the patient. The dose may be repeated at an appropriate frequency which may be in the range of once or twice per day, once or twice per week to once every three months, depending on the pharmacokinetics of the polypeptide composition (e.g., half-life in the circulation) and the pharmacodynamic response (e.g., the duration of the therapeutic effect).

Administration can be periodic. Depending on the route of administration, the dose can be administered, e.g., once every 1, 3, 5, 7, 10, 14, 21, or 28 days or longer (e.g., once every 2, 3, 4, or 6 months). In some cases, administration is more frequent, e.g., 2 or 3 times per day. The patient can be monitored to adjust the dosage and frequency of administration depending on therapeutic progress and any adverse side effects.

Dosages can be empirically determined considering the type and severity of cognitive condition diagnosed in a particular patient. The dose administered to a patient, in the context of the present disclosure, should be sufficient to affect a beneficial therapeutic response in the patient over time. The size of the dose will also be determined by the existence, nature, and extent of any adverse side-effects that accompany the administration of any particular composition in a particular patient, as will be recognized by the skilled practitioner.

In some embodiments, the polypeptide composition is administered to an (e.g., human) individual having at least normal cognitive function. As described herein, it has been surprisingly shown that a protein of Table 1 or Table 2 can improve cognition of individuals with at least normal cognition. Thus in some embodiments, the individual receiving a polypeptide composition of Table 1 or Table 2 begins initially with at least normal cognition and following administration of the polypeptide composition attains improved cognition compared to the initial level of cognition. The level of cognition of an individual can be determined as is known in the art. Normal cognitive functions are determined by scores from sets of cognitive tests that are compiled into global cognitive scores, as described in Dubal D B et al. (2014) Cell Reports 7:1065-1076. Such cognition tests include tests of executive function and working memory such as Trails A and Trails B (Dubal D B et al. (2014) Cell Reports 7:1065-1076). In some embodiments, administration of the polypeptide results in an improvement of cognition (whether initially at least normal or impaired), by at least 5%, 10%, 20% or more.

In some embodiments, administration results in improved motor function. In some embodiments, the polypeptide composition is administered to an (e.g., human) individual having impaired motor function. For example, in some embodiments, the individual has stroke to the brain or spinal cord (ischemic or hemorrhagic), neurodegenerative disease (Parkinson's disease, Lewy body dementia, multiple system atrophy, amyotropic lateral sclerosis, prion disorder, Huntington's disease, supranuclear palsy), Parkinsonism, traumatic brain injury, neuroinfectious brain lesions, multiple sclerosis and related autoimmune and demyelinating disease, spinal cord lesions (compressive, infectious, toxic or metabolic, autoimmune, oncologic), brain tumor, epilepsy, paraneoplastic disorder, neurodevelopmental disorder (mitochondrial, autosomal genetic), muscle disease (polymyositis, dermatomyositis, inclusion body myositis, infectious, endocrine, metabolic, toxic, congenital myopathy, congential muscular dystrophy, hereditary), neuropathies (Guillain-Barre syndrome, axonal and demyelinating, diabetic, toxic, metabolic, infectious, critical illness, entrapment), tick paralysis, myasthenia gravis, and spinal muscular atrophy. Changes in motor function can be assayed as known in the art. Exemplary motor function assays include but are not limited to electromyogram and nerve conduction studies, direct or device-assisted clinical testing of strength, tone, and muscle bulk, reflex examination, coordination examination, and gait analysis. Assays for testing etiologies causing deficits of motor function include but are not limited to magnetic resonance imaging of the central nervous system, muscle biopsy, nerve biopsy, and laboratory studies.

Thus in some embodiments, additional administration is dependent on patient progress, e.g., the patient is monitored between administrations. For example, after the first administration or round of administrations, the patient can be monitored for cognitive ability or for side effects, e.g., weakness, dizziness, nausea, etc.

In some embodiments, the individual has a chronic condition, so that the polypeptide is administered over an indefinite period, e.g., for the lifetime of the patient. In such cases, administration is typically periodic. Diseases that are considered long-term or chronic include, but are not limited to Alzheimer's disease, Parkinson's disease, Huntington's disease, and cognitive decline associated with hypertension and heart disease.

In some embodiments, the polypeptide is linked to a stabilizing moiety such as PEG, glycosylation, or a liposome or other nanocarrier. U.S. Pat. Nos. 4,732,863 and 7,892,554 and Chattopadhyay et al. (2010) Mol Pharm 7:2194 describe methods for attaching a polypeptide to PEG, PEG derivatives, and nanoparticles (e.g., liposomes). Liposomes containing phosphatidyl-ethanolamine (PE) can be prepared by established procedures as described herein. The inclusion of PE provides an active functional site on the liposomal surface for attachment. In some embodiments, the polypeptide is linked to an affinity tag, e.g., a histidine tag (e.g., 4-16 contiguous histidine residues), streptavidin, or an antibody target.

The polypeptide can also be formulated as a sustained-release preparation (e.g., in a semi-permeable matrices of solid hydrophobic polymers (e.g., polyesters, hydrogels (for example, poly (2-hydroxyethyl-methacrylate), or poly (vinylalcohol)), polylactides. The polypeptide can be entrapped in a nanoparticle prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nanoparticles and nanocapsules) or in macroemulsions.

In some embodiments, the polypeptide is labeled, e.g., for tracking in the body or ex vivo. The polypeptide can be labeled any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). The diagnostic agent can be detected by a variety of ways, including as an agent providing and/or enhancing a detectable signal. Detectable signals include, but are not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. The terms “detectable agent,” “detectable moiety,” “label,” “imaging agent,” and like terms are used synonymously herein.

In some embodiments, the label can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives. Fluorescent dyes are discussed, for example, in U.S. Pat. Nos. 4,452,720, 5,227,487, and 5,543,295.

The label can also be a radioisotope, e.g., radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In some embodiments, radioactive agents can include ¹¹¹In-DTPA, ^{99m}Tc(CO)₃-DTPA, ^{99m}Tc(CO)₃-ENPy₂, ^{62/64/67}Cu-TETA, ^{99m}Tc(CO)₃-IDA, and ^{99m}Tc(CO)₃triamines (cyclic or linear). In some embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, a nanoparticle can be labeled by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l I Pharmaceutics 344:110-117 (2007).

In some embodiments, the diagnostic agent can be associated with a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Secondary binding ligands include, e.g., biotin and avidin or streptavidin compounds.

Cognitive Conditions and Disorders

Polypeptides of Table 1 or Table 2 (and functional variants and fragments thereof) can be administered to improve cognition for a number of conditions and situations. This includes treatment of individuals with lower than normal or declining cognitive ability, or prophylactic treatment of individuals in need of improved or increased cognitive ability.

The polypeptides (and functional variants and fragments thereof) can be used to prevent or reduce cognitive decline associated with aging, e.g. in individuals 50 years of age or older, or upon initial signs of cognitive decline.

The polypeptides (and functional variants and fragments thereof) can also be used to treat individuals with age-related, non-age related, or disease related conditions including, but not limited to:

Neurodegenerative diseases and dementia: Alzheimer's disease, Parkinson's disease, Huntington's disease, frontotemporal dementia, progressive supranuclear palsy, corticobasalar degeneration, mild cognitive impairment, vascular dementia, Lewy body dementia, amyotropic lateral sclerosis, prion disorder, HIV-related dementia;

Mental or mood disorders: depression, schizophrenia, attention deficit/hyperactivity disorder, autism spectrum disorder, intellectual disability, a mood disorder, and a psychotic disorder;

Childhood neurodevelopmental syndromes and brain tumors: X-linked mental disability or retardation, astrocytoma, ependymoma, medulloblastoma, oligodendroglioma;

Genetic syndromes affecting learning: Down's syndrome, Angelman's syndrome, Rett's syndrome;

Metabolic disorders affecting cognition: phenylketonuria, Lesch-Nyhan, galactosemia, and adrenoleukodystrophy;

Cognitive decline associated with chemotherapy and/or radiation therapy; and

Additional conditions and disorders: pain-associated cognitive effects, traumatic brain injury, stroke, multiple sclerosis, neuroautoimmune disease, epilepsy, delirium, paraneoplastic disorder, developmental delay, and leukodystrophies.

The polypeptides (and functional variants and fragments thereof) can be also be administered to provide increased cognition for individuals desiring improved cognition, e.g., individuals exposed to stress, sleep deprivation, or jet lag, or for individuals requiring superior cognitive function, such as surgeons, air-traffic controllers, and military personal. In such cases, the polypeptide composition can be administered 2-24 hours before the desired effect, which can last about 3-5 days for working memory and about 2 weeks for spatial memory.

Cognitive ability can be measured using any method known in the art, e.g., for testing memory, language ability, executive functions, visuospatial function, dementia, or multi-parameter neuropsychological abilities. In some embodiments, polypeptide administration results in at least a 1%, 2%, 5%, 7%, 10%, 15%, 20%, 30%, 50%, or greater improvement in score on a standard cognitive ability test (e.g., measured 1-3 days after administration). In some embodiments, the testing is carried out more than once for an individual, e.g., one or more time over the course of treatment with the polypeptide.

For example, standard tests for memory and learning can be applied, e.g., to determine semantic, episodic, procedural, priming, and/or working (i.e., short term) memory. Common tests include Cambridge prospective memory test (CAMPROMPT), memory assessment scales (MAS), Rey auditory verbal learning test, Rivermead behavioral memory test, Test of memory and learning (TOMAL), Wechsler memory scale (WMS), and Test of memory malingering (TOMM). Tests for language functions include, e.g., Boston Diagnostic Aphasia Examination (BDAE), Comprehensive aphasia test (CAT), and Multilingual aphasia examination (MAE).

Executive function (e.g., problem solving, planning, organization, inhibitory control) can be tested using Behavioral assessment of dysexecutive syndrome (BADS), CNS vital signs (Brief Core Battery), Controlled oral word association test (COWAT), Delis-Kaplan Executive Function System (D-KEFS), Digit vigilance test, Kaplan Baycrest neurocognitive assessment (KBNA), Hayling and Brixton tests, Tests of variables of attention (TOVA), Wisconsin card sorting test (WCST), or Test of everyday attention (TEA). Visuospatial ability (e.g., visual perception, construction and integration) can be tested using the Clock Test, Hooper visual organization task (VOT), or Rey-Osterrieth complex figure tests. Dementia can be quantified using the clinical dementia rating or dementia rating scale.

Multi-parameter tests for neuropsychological function (e.g., cognitive function) include but are not limited to the Barcelona neuropsychological test (BNT), Cambridge neuropsychological test automated battery (CANTAB), Cognistat, Cognitive assessment screening instrument (CASI), Cognitive function scanner (CFS), Dean-Woodcock neuropsychology assessment system (DWNAS), General practitional assessment of cognition (GPCOG) Mini mental state examination (MMSE), NEPSY, or the CDR computerized assessment system.

Alternatively, cognition can be determined using structural or molecular proxies for cognitive activity, e.g., compared over time to detect changes. Cognitive changes can be detected, e.g., by observing changes to brain structure, connectivity, activation, inhibition, or synaptic plasticity, e.g., by MRI, fMRI, EEG, TMS and TES, and/or any combination of these. In some embodiments, brain activity is observed. In some embodiments, polypeptide administration results in a 1.5-fold, 2-fold, 5-fold, 7-fold, 10-fold, or greater increase in brain activity (e.g., measured 1-3 days after administration). Molecular proxies for improved cognition include, but are not limited to: increased levels of GluN2B, increased GluN2B synaptic localization, increased NMDA receptor activation, and/or increased c-fos activation in the brain. These measures are particularly relevant to cognition. Such method can include, e.g., obtaining a sample of neuronal tissue or CSF from an individual and using standard assays to determine gene expression or activation.

Similarly, in mice and other non-human animals, cognitive ability can be tested with measures of executive function (working memory, attention, processing speed, set shifting), visiospatial learning and memory, object memory, pattern recognition, fear memory, passive avoidance memory, habituation, and novel object recognition, for example. Common tests include but are not limited to the Morris water maze, Barnes maze, radial arm water maze, y-maze, T-maze, and open field habituation. Brain imaging techniques are similarly applicable.

### EXAMPLES

### Example 1

Methods

Mice

All studies were conducted in a blinded manner in C57BL/6 mice. Young mice and aged mice were obtained from The Jackson Laboratory and the National Institute on Aging (NIA) mouse colonies, respectively. Mice were randomly assigned to each group, and the experimenter was blinded to their treatment. Mice were kept on a 12-hr light/dark cycle with ad libitum access to food (Picolab Rodent Diet 20) and water. All studies were approved by the Institutional Animal Care and Use Committee of the University of California, San Francisco and conducted in compliance with NIH guidelines.

Plasma Profiling of Mouse Plasma

Mouse klotho (R&D, 1819-KL) was diluted in PBS (pH7.5) and administered as an i.p. injection at a volume of 10 ul/gram (adjusted to weight of mouse) at a dose of 10 ug/kg. All young male mice (4 months old) were injected with vehicle or klotho (n=10 mice per group). Four hours later, they explored a small Y-maze for 10 minutes and their brains were immediately harvested following anesthesia with avertin (i.p.). Whole blood was collected from the cardiac puncture route into EDTA-coated tubes (Sastedt), centrifugated with 10,000 rpm for 10 min and then plasma was transferred to a low-binding tube (Sastedt). Plasma samples were processed for analyzed by mass spectrometry at Biognosys, Zurich, Switzerland.

Cognitive Behavioral Test

Mouse platelet factor 4 (PF4) (PROSPEC, chm-245) was diluted in PBS (pH7.5) and administered as an i.p. injection at a volume of 10 ul/gram (adjusted to weight of mouse) 1 h before each day of training and testing at a dose of (20 ug/kg). All female aged mice (18-21 months old, n=8 mice per group) were tested in two-trials Y-maze as described in Dellu F, Mayo W, Cherkaoui J, Le Moal M, Simon H. A two-trial memory task with automated recording: study in young and aged rats. Brain Res. 1992; 588(1):132-139. Briefly, mice underwent training by exploring the maze with a visual cue in one arm and another arm blocked off. 16 h after training, mice underwent testing with the all three arms open (start arm, familiar arm, novel arm) and the time spent exploring the novel arm compared to the familiar arm, an index of memory, was tested.

Results

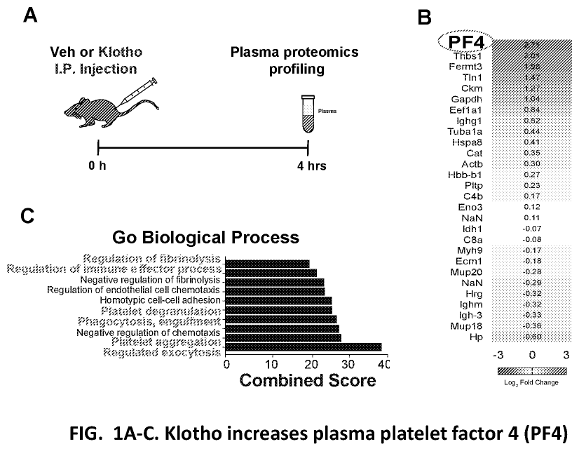

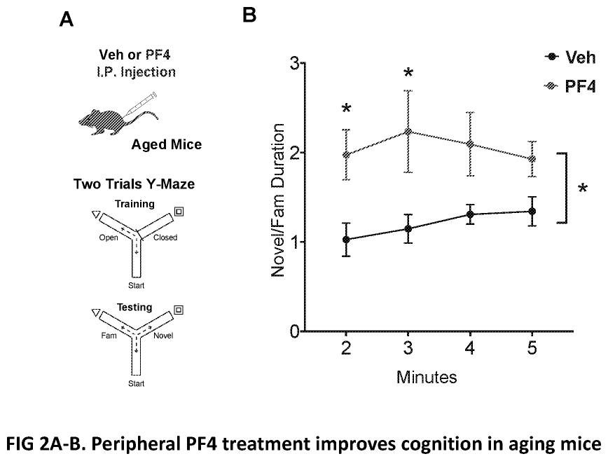

In order to assess how systemic elevation of klotho in the body sends a signal to boost cognition, we profiled plasma proteins following systemic klotho treatment (FIG. 1A-C). Klotho significantly increased several plasma platelet factors (FIG. 1B), indicating a novel biologic action of klotho in inducing platelet activation and function (FIG. 1C). Klotho treatment most robustly increased platelet factor 4 (PF4) (FIG. 1B), a pleiotropic chemokine that increases with exercise and enhances neurogenesis (Leiter O, Seidemann S, Overall R W, et al. Exercise-Induced Activated Platelets Increase Adult Hippocampal Precursor Proliferation and Promote Neuronal Differentiation. Stem Cell Reports. 2019; 12(4):667-679). We then tested whether PF4 itself can cognition in the aging brain (FIGS. 2A and B). Indeed, systemic treatment with recapitulated klotho-mediated improvement of cognition (FIG. 2B). These findings collectively suggest that klotho increases platelet factor, such as PF4—and factors such as PF4 induce cognitive enhancement.

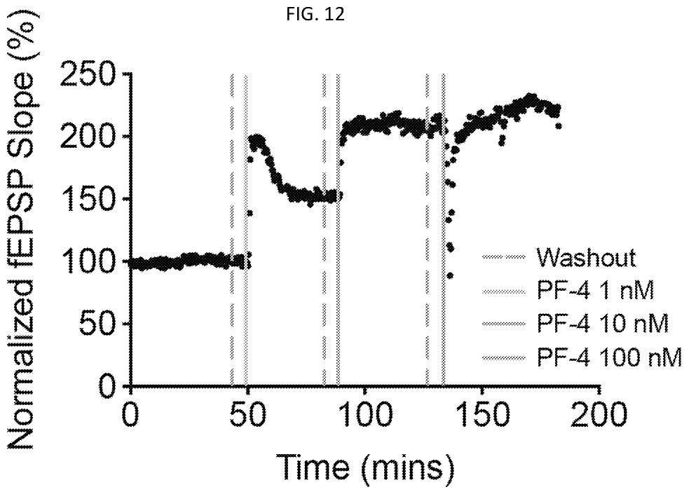

Further Methods (FIG. 12): Coronal brain slices of 300 um thickness from 3 month old mice were obtained as described with some modifications (Dubal D B, Yokoyama J S, Zhu L, et al. Life extension factor klotho enhances cognition. Cell Rep. 2014; 7(4):1065-1076; Dubal D B, Zhu L, Sanchez P E, et al. Life extension factor klotho prevents mortality and enhances cognition in hAPP transgenic mice. J Neurosci. 2015; 35(6):2358-2371) including that measurements were obtained from the CA1 region following stimulation of the Schaffer Collateral path. Wild-type C57BL/6J mice were anesthetized with isofluorane and decapitated. The brain was harvested and immediately placed in ice-cold artificial cerebrospinal fluid (aCSF) containing the following (in mM): 124 NaCl, 2.8 KCl, 2 MgSO₄, 1.25 NaH₂PO₄, 10 Glucose, 26 NaHCO₃, 2.5 CaCl₂, 1.3 Ascorbic acid and sliced on a vibratome (Leica). Slices were incubated at 32° C. for 30 minutes, then recovered at RT for 1 hour prior to testing. Slices were transferred to an interface chamber with circulating oxygenated (95% O₂ and 5% CO₂) aCSF at 30° C. and left to recover for 10-15 minutes prior to any stimulation.

For field potential recordings, acute hippocampal slices were placed on a Med64-Quad II multielectrode array (Alpha MED Scientific), which enables recording of 4 slices simultaneously. Field Excitatory Post Synaptic Potentials (fEPSP) were elicited and recorded via planar electrodes of the Quad II 2×8 Probe AL-MED-PG501A by aligning the electrodes and the stratum radiatum region of hippocampal slices. An input-output curve was performed at the beginning of each recording to determine the appropriate stimulation intensity. Test stimuli at 30-40% of maximal intensity were delivered at 0.05 Hz. A stable baseline was recorded for at least 30 mins and then following a brief washout period, PF-4 was added to the bath at concentrations of 1 nM, 10 nM, and 100 nM, respectively. The slices were monitored for at least 30 minutes following application of PF-4.

Further Results (FIG. 12): PF4 addition to hippocampal slices increased synaptic plasticity in a dose-dependent manner. This measure is important because synaptic plasticity is a cellular and molecular substrate that underlies learning and memory. These preliminary data, which require replication, suggest that PF4 (which is predicted to cross the blood brain barrier) acts directly in the central nervous system to increase or modulate neuronal activity that is important to cognition.

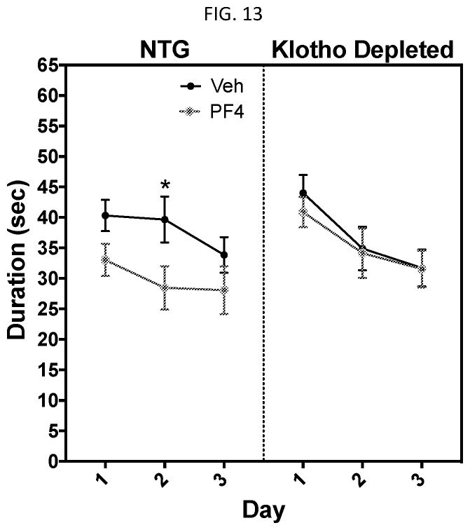

Further Methods and Results (FIG. 13). Mice were treated daily with PF4 (20 μg/kg, ip) during the water maze testing. Following training in the hidden maze as described (Dubal D B, Yokoyama J S, Zhu L, et al. Life extension factor klotho enhances cognition. Cell Rep. 2014; 7(4):1065-1076; Dubal D B, Zhu L, Sanchez P E, et al. Life extension factor klotho prevents mortality and enhances cognition in hAPP transgenic mice. J Neurosci. 2015; 35(6):2358-2371) the platform was “reversed” or changed to a new location. Following this reversal, PF4 increased cognition in young mice (n=15/group; age 4 mos). PF4-induced cognitive enhancement was dependent upon klotho. That is, in klotho-depleted mice (via genetic suppression as described in Kuro-o M, Matsumura Y, Aizawa H, et al. Mutation of the mouse klotho gene leads to a syndrome resembling ageing. Nature. 1997; 390(6655):45-51), the ability of PF4 to improve cognition was blocked. These data show that:

1. PF4 can enhance cognition in young mice

2. The PF4-mediated enhancement depended upon klotho.

### Example 2

Results

Aging changes the adult brain at the molecular and cellular levels, driving cognitive impairments and increasing susceptibility to neurodegenerative diseases. Systemic rejuvenating interventions, such as heterochronic parabiosis (in which the circulatory systems of young and old mice are joined), improve synaptic plasticity and cognition in aged mice. The plasma component of blood is particularly effective at reversing neuronal and hippocampal-dependent cognitive impairments in aged mice Enhancements elicited by exposure to young blood are mediated, in part, by activation of the cAMP response element binding protein (Creb) in the aged hippocampus (Villeda, S. A., Plambeck, K. E., Middeldorp, J., Castellano, J. M., Mosher, K. I., Luo, J., . . . Wyss-Coray, T. (2014). Young blood reverses age-related impairments in cognitive function and synaptic plasticity in mice. Nature Medicine, 20(6), 659-663). Therefore, we sought to utilize Creb phosphorylation to screen for prospective novel pro-youthful components of young blood plasma.

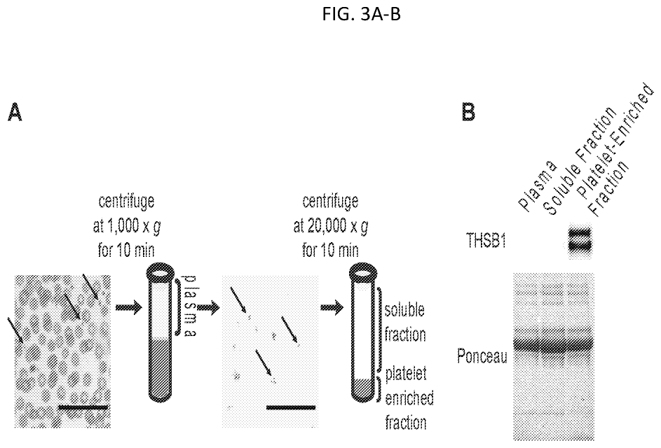

Using a centrifugation-based fractionation approach we have identified platelets within a fraction of young blood plasma (FIG. 3A). Specifically, following collection, plasma was centrifuged at 20,000 g for 10 min at 4° C. The supernatant was collected as the soluble fraction of plasma and the pelleted component was resuspended in an equivalent volume of saline. Western blot analysis revealed Thrombospondin-1 (THSB1), a platelet marker, to be enriched within the pelleted fraction (FIG. 3B).

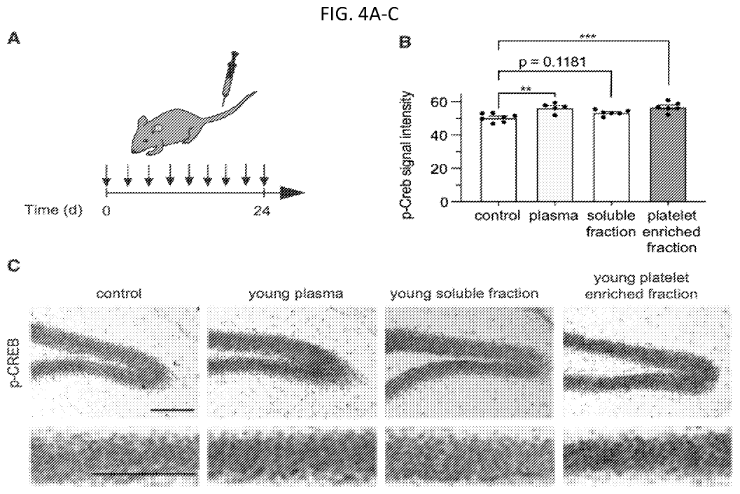

To screen these distinct components of young blood plasma for the potential to rescue age-related impairments in the hippocampus, we examined phosphorylated Creb in the dentate gyrus (DG) of aged mice (20 months) systemically treated with saline, plasma, the soluble fraction of plasma, or the platelet-enriched fraction of plasma from young mice (2 months). The injection volumes and timelines for each treatment were selected to recapitulate those previously shown to demonstrate the rejuvenating potential of young blood plasma administration on the aged hippocampus (FIG. 4A) (Villeda, S. A., Plambeck, K. E., Middeldorp, J., Castellano, J. M., Mosher, K. I., Luo, J., . . . Wyss-Coray, T. (2014). Young blood reverses age-related impairments in cognitive function and synaptic plasticity in mice. Nature Medicine, 20(6), 659-663). Creb phosphorylation significantly increased in the DG after administration of both young plasma and the platelet-enriched fraction of young plasma, whereas treatment with the soluble fraction did not produce a significant increase (FIG. 4B-C). These data suggest that the platelet-enriched fraction of young plasma may be capable of recapitulating, at least in part, the rejuvenating effect of young plasma on the aged hippocampus.

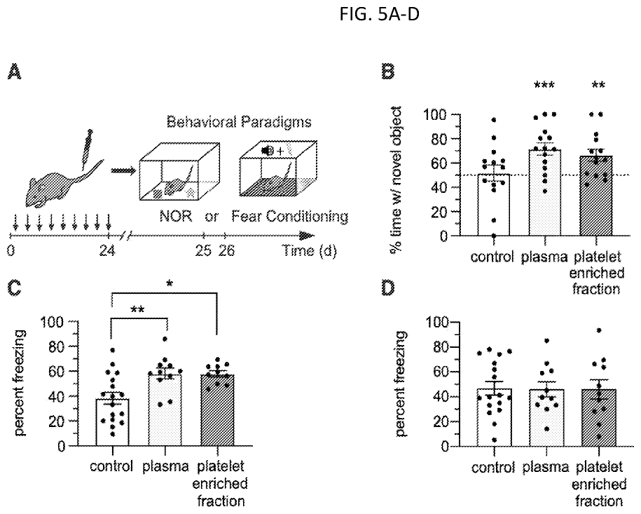

To assess the potential of the platelet-enriched fraction of young plasma to rescue age-related impairments in hippocampal-dependent learning and memory, aged mice were systemically administered either saline, young plasma, or the platelet-enriched fraction of young plasma prior to cognitive testing (FIG. 5A). Hippocampal-dependent cognitive function was assessed using the Novel Object Recognition (NOR) and contextual fear conditioning paradigms. During NOR testing, aged mice treated with young plasma and the platelet-enriched fraction of young plasma spent significantly more time with a novel object relative to a familiar object, while saline treated mice showed no preference for the novel object (FIG. 5B). Systemic administration of the platelet-enriched fraction of young plasma to aged mice also increased freezing behavior in the contextual (FIG. 5C), but not cued (FIG. 5D), memory testing relative to saline treated mice, similar to the increase found with young plasma treatment. These data indicate that blood factors within the platelet-enriched fraction of young plasma are sufficient to ameliorate impairments in hippocampal-dependent learning and memory in aged mice.

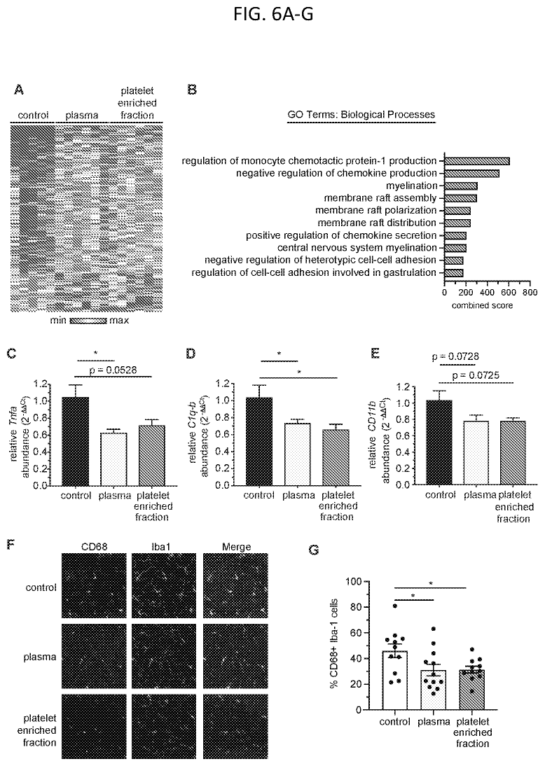

To better understand the mechanisms whereby the young platelet-enriched fraction of plasma rejuvenates the aged hippocampus we performed RNA-seq on bulk hippocampal tissue from aged mice following systemic administration of saline, young plasma, or the platelet-enriched fraction of young plasma. We compared overlapping significantly differentially expressed genes from mice treated with young plasma and the platelet-enriched fraction of young plasma, relative to saline treated mice (FIG. 6A). We utilized this list of overlapping upregulated genes to identify Biological Processes Gene Ontology (GO) terms to better understand mechanisms whereby the platelet-enriched fraction of young plasma recapitulates the rejuvenating potential of young plasma (FIG. 6B). Interestingly, we discovered a number of terms associated with regulation of immune function. In a separate cohort of aged mice treated with saline, young plasma, or the platelet-enriched fraction of young plasma, we found a reduction in expression of inflammation-related genes (Tnfa, C1q-b, and CD11b) in the hippocampus (FIG. 6C-E). Additionally, hippocampal microglial activation was analyzed by Ibal and CD68 immunolabeling (FIG. 6F). Aged mice treated with either young plasma or the platelet-enriched fraction of young plasma had a reduced percentage of CD68-positive/Ibal-positive microglia, suggesting reduced activation of microglia within the DG (FIG. 6G). These data suggest that blood factors within the platelet-enriched fraction of young plasma may promote rejuvenation of the aged hippocampus via mitigation of age-related inflammation.

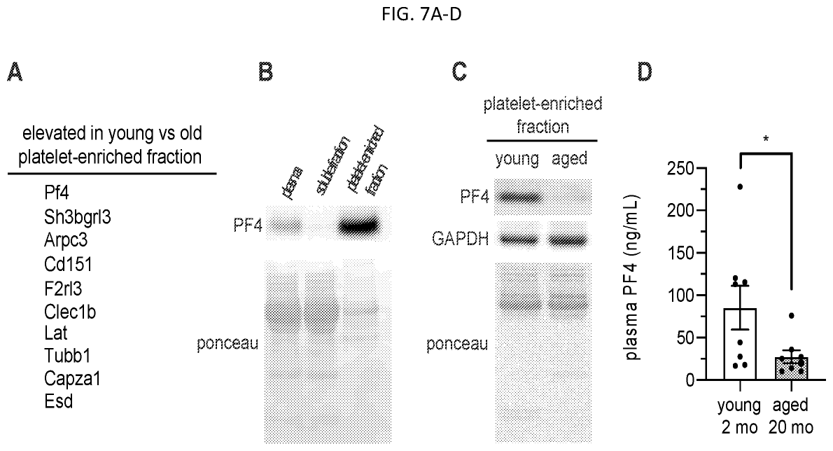

To begin identifying factors potentially responsible for the young platelet-mediated rejuvenation of hippocampal function in aged mice we utilized a proteomic mass spectrometry approach to identify proteins elevated in the platelet-enriched fraction of plasma from young mice relative to aged mice. The top 10 proteins enriched in the platelet fraction from young relative to old mice are displayed (FIG. 7A). Most prominent among these potential platelet-derived pro-youthful circulating factors was Platelet Factor-4 (PF4). PF4 is a chemokine that is released from platelets and has been shown to have a variety of immunomodulatory functions (Eisman, R., Surrey, S., Ramachandran, B., Schwartz, E., & Poncz, M. (1990). Structural and functional comparison of the genes for human platelet factor 4 and PF4alt. Blood, 76(2), 336-344). Western blot analysis revealed that PF4 is enriched in the platelet-enriched fraction relative to young plasma, and is undetectable in the soluble fraction of young plasma (FIG. 7B). Additionally, PF4 was greatly reduced in the platelet-enriched fraction of plasma from aged mice relative to young mice, corroborating our mass spectrometry data (FIG. 7C). An ELISA revealed a reduction in PF4 in whole plasma from aged relative to young mice (FIG. 7D). Highlighting the translational potential of PF4, an age-dependent decrease in expression has also been shown at the transcript level in human platelets (Simon, L. M., Edelstein, L. C., Nagalla, S., Woodley, A. B., Chen, E. S., Kong, X., . . . Bray, P. F. (2014). Human platelet microRNA-mRNA networks associated with age and gender revealed by integrated plateletomics. Blood, 123(16), 37-46. https://doi.org/10.1182/blood-2013-12-544692).

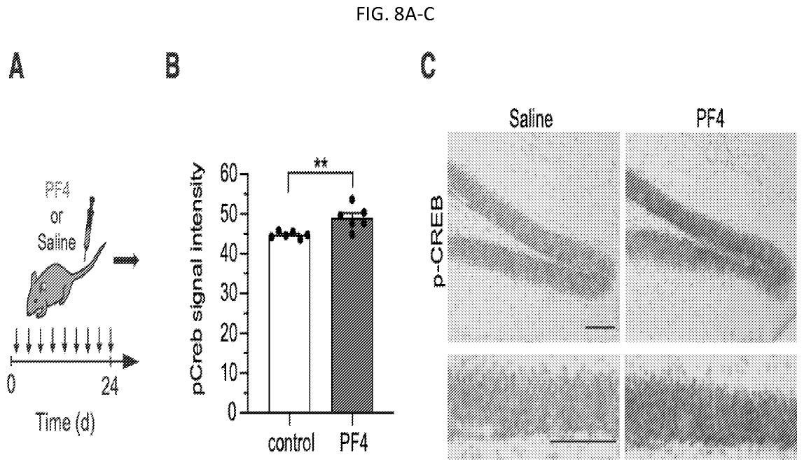

To begin to assess whether PF4 may function as a pro-youthful platelet-derived factor we examined phosphorylated Creb in the DG of aged male mice (20 months) systemically treated with saline or recombinant PF4 (FIG. 8A). Creb phosphorylation significantly increased in the DG following PF4 administration (FIG. 8B-C), suggesting PF4 may function as a pro-youth platelet-derived factor capable of rejuvenating the aged hippocampus.

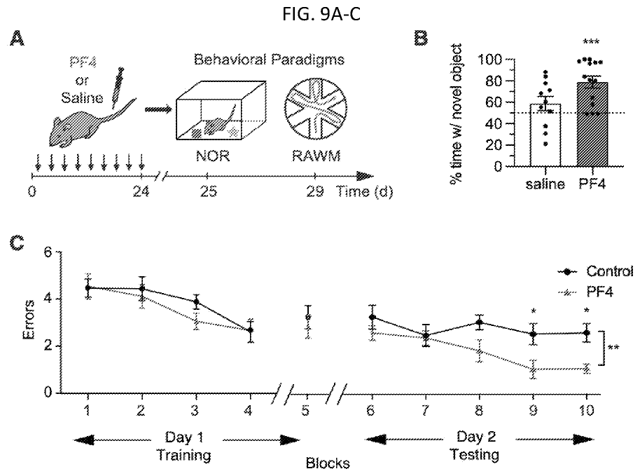

To assess the potential of PF4 to rescue age-related impairments in hippocampal-dependent learning and memory, aged mice were systemically administered either saline or PF4 prior to cognitive testing (FIG. 9A). Hippocampal-dependent cognitive function was assessed using the NOR and radial arm water maze (RAWM) paradigms. During NOR testing, aged mice treated with PF4 spent significantly more time with a novel object relative to a familiar object, while saline treated mice showed no preference for the novel object (FIG. 9B). In the training phase of the RAWM paradigm all mice showed similar spatial learning capacity (FIG. 9C). However, aged animals administered PF4 demonstrated improved learning and memory for the platform location during the testing phase of the task compared to aged saline treated controls (FIG. 9C). These data indicate that systemic administration of PF4 can ameliorate impairments in hippocampal-dependent learning and memory in aged mice.

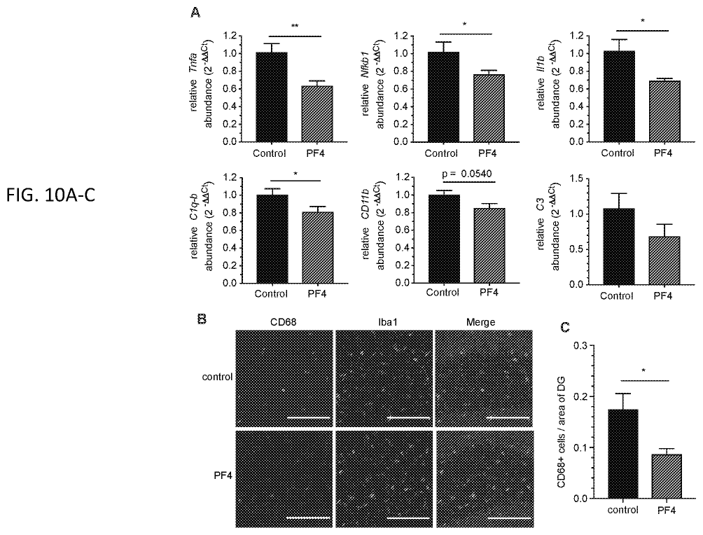

To better understand the mechanism whereby PF4 may rejuvenate the aged hippocampus we assessed the expression level of inflammation-related genes in the hippocampus of aged mice treated with saline or PF4. We found hippocampi from PF4 treated mice had reduced expression of genes associated with inflammatory cytokines (Tnfa, Nfkb, and Il1b), the complement cascade (Clq-b and C3), and microglial activation (CD11b) (FIG. 10A). Additionally, hippocampal microglial activation was analyzed by Ibal and CD68 immunolabeling (FIG. 10B). Aged mice treated with PF4 had a reduced number of CD68-positive microglia, suggesting a reduction in the number of activated microglia within the DG (FIG. 10C). These data suggest that PF4 may promote rejuvenation of the aged hippocampus via mitigation of age-related inflammation.

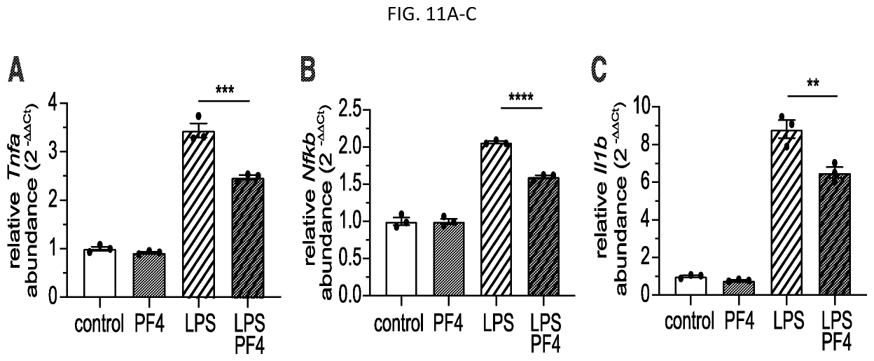

To understand whether PF4 can directly act upon microglia to mitigate inflammation, we utilized the BV2 microglial cell culture model. Cells treated with lipopolysaccharide (LPS) had a large induction of expression of genes associated with inflammatory cytokines (Tnfa, Nfkb, and Il1b); however, PF4 significantly inhibited the LPS-induced induction of inflammatory genes (FIG. 11A-C). These data suggest that systemic administration of PF4 may directly act upon microglia to attenuate inflammation, thereby promoting rejuvenation of the aged hippocampus. Collectively, these data identify platelets and PF4 as cellular and molecular components of young blood sufficient to rejuvenate hippocampal and cognitive function in aged animals.

### Example 3

Some information presented in Example 1 and Example 3 is based on the same experiments.

Platelet factors regulate wound healing and also signal from the blood to the brain. However, whether platelet factors modulate cognition, a highly valued and central manifestation of brain function, is unknown. Here, we show that systemic platelet factor 4 (PF4) enhances cognition in the young and aging mouse brain. Klotho, a longevity and cognition-enhancing protein, acutely activated platelets and increased circulating platelet factors, most robustly platelet factor 4 (PF4). Transgenic mice overexpressing PF4 along with platelet basic protein increased long-term potentiation (LTP), a form of synaptic plasticity and underlying substrate of learning and memory. Blockade of NMDA receptor subunit GluN2B, with key functions in synaptic plasticity and learning and memory, abolished the platelet factor effects. To specifically and directly test PF4 effects on LTP and on cognition, we treated mice with vehicle or systemic PF4. PF4 treatment alone was sufficient to enhance LTP. Further, PF4 increased cognition in young mice and reversed cognitive deficits in aging mice. Augmenting platelet factors such as PF4, a possible messenger of klotho from the blood to the brain, may enhance cognition and counteract effects of cognitive aging in the brain.

Systemic klotho treatment activated platelets and treatment with klotho-induced platelet factor 4 (PF4) enhanced cognition in young and aging mice.

Platelets are small, anuclear blood cells that store bioactive factors in specialized cytoplasmic compartments (1). Upon environmental stimulation such as exercise, tissue injury, or stress, varying doses and types of platelet activation cause context-dependent and selective release of contents. Thus, diverse forms of platelet activation transduce fundamental biologic actions ranging from hemostasis to neurogenesis (2). Likewise, platelet dysfunction is implicated in inflammation, bleeding, and CNS diseases (3). The idea that platelets could be messengers of brain health is supported by observations that exercise activates platelets and subsequent release of platelet factor 4 (PF4) increases hippocampal neurogenesis (2). However, whether platelet factors could modulate cognition itself, a highly valued and central manifestation of brain function that declines with aging and disease, is unknown. This is an important knowledge gap since cognitive dysfunction is among our biggest biomedical challenges with no effective treatments. We thus investigated platelet factor function on underlying substrates of cognition, and on cognition itself.

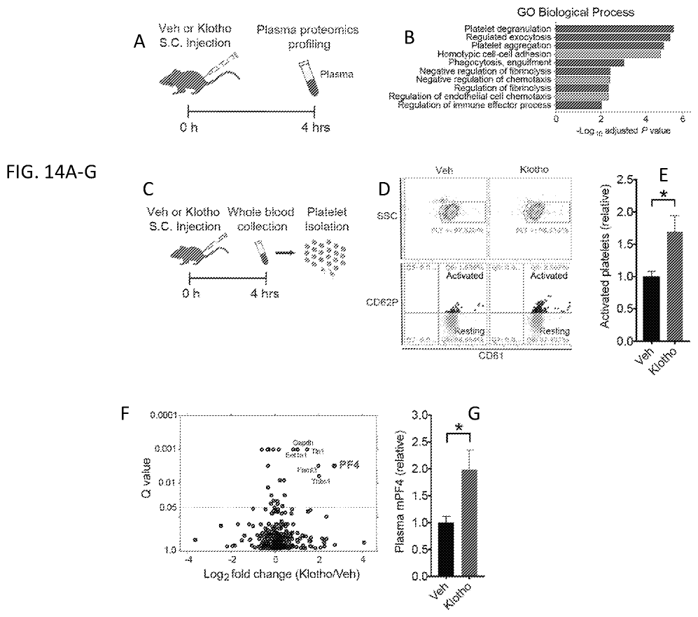

An unbiased proteomic analysis identified platelet factor biology as a target of klotho, a longevity factor (4-6) that enhances cognitive functions (7-12) without crossing into the brain (10, 13). Human genetic variation of KLOTHO increases its systemic levels (7, 14) and associates with enhanced brain connectivity (14) and cognition (7, 14, 15) in aging human populations. Similarly, acute and systemic elevation of the secreted form of α-klotho (klotho) in mice increases synaptic plasticity, cognition, and neural resilience (10). Since peripherally injected klotho does not cross into the brain, we hypothesized that klotho engages peripheral messengers that transduce signals into the brain. To identify klotho-mediated cognitive signals, we performed an unbiased mass spectrometry-based proteomic profiling of plasma isolated from mice 4 h following peripheral treatment with vehicle or klotho and a cognitive task, exploration of a small Y maze (FIG. 14A). Biological pathway analysis of the plasma proteome from vehicle-compared to klotho-treated mice revealed the marked enrichment of platelet functions (FIG. 14B). These findings suggested that systemic klotho could influence platelets.

We next directly tested whether systemic klotho treatment can activate platelets. Mice underwent vehicle or klotho treatment; 4 h later, following exploration of a small Y-maze, platelets were immediately isolated from whole blood and sorted by fluorescence-activated cell sorting (FACS) (FIG. 14C). We analyzed and quantified platelet activation levels by flow cytometry (3, 16), expressed as the percentage of activated platelets (CD62P-positive) within the total platelet population (CD61-positive). Acute klotho treatment followed by a cognitive task doubled the resting level of platelet activation. (FIGS. 14D and E). It will be important to define how this district form of klotho-induced platelet activation could parallel others, including that induced by exercise.

Quantitative analysis of plasma proteomics identified that klotho increased several platelet factors resulting from platelet activation and subsequent α-granule release (FIG. 14F). Among these, PF4 was selectively expressed at the highest level. We validated the proteomic finding that acute klotho treatment increased PF4 in the plasma using enzyme linked immunoassay (ELISA) (FIG. 14G).

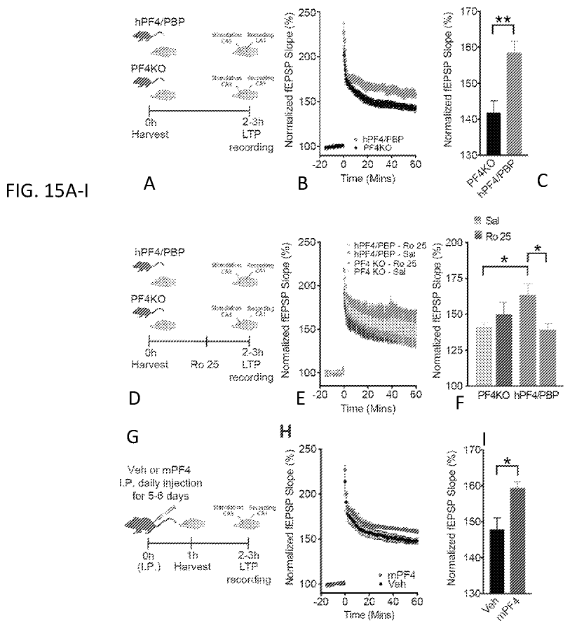

We next tested whether transgenic overexpression of platelet factors, in parallel with effects of transgenic overexpression of klotho (7), could increase synaptic plasticity in the form of long term potentiation (LTP), an NMDA receptor-dependent and excitatory substrate of learning and memory (17, 18) (FIG. 15A). We assessed LTP in acute hippocampal slices in the CA1 Schaffer collateral pathway synapse in transgenic mice overexpressing human PF4 at 6-times baseline levels (19) along with human platelet basic protein (PBP) on a mouse PF4 knockout background (20). Thus, similar to klotho, PF4/PBP elevation enhanced LTP (FIG. 15B, 15C).

Synaptic plasticity in this form of LTP is largely NMDAR-dependent. Since klotho augments the GluN2B contribution to NMDAR signaling (7, 8, 10) and platelet factors capitulated klotho-mediated synaptic enhancement, we tested whether blocking GluN2B-containing NMDARs modulates platelet factor effects on synaptic plasticity. To this end, we used Ro 25-6981 (Ro 25) a GluN2B-specific antagonist with 3000-fold specificity to GluN2B compared to other NMDAR subunits (21, 22). Acute hippocampal slices from PF4/PBP overexpressing, transgenic mice were treated with either vehicle or a low dose of Ro 25 (1.5 μM). As anticipated, Ro 25 did not alter LTP in control PF4KO mice at the low dose (FIG. 15E, 15F). In contrast, low dose Ro 25 completely abolished the platelet factor-induced enhancement of LTP (FIG. 15E, 15F). Taken together, these findings indicate that platelet factors engage glutamatergic signaling to enhance LTP, a key substrate of learning and memory.

To delineate and test directly whether platelet factor PF4, itself, is sufficient to enhance LTP, mice were treated daily for 5-6 days with vehicle or systemic (20 μg/kg i.p.) mouse PF4 (FIG. 15G). Indeed, PF4 treatment was sufficient, in the absence of other platelet factors, to enhance LTP determined by field excitatory postsynaptic potentials (fEPSP) recordings (FIG. 15H, 15I).

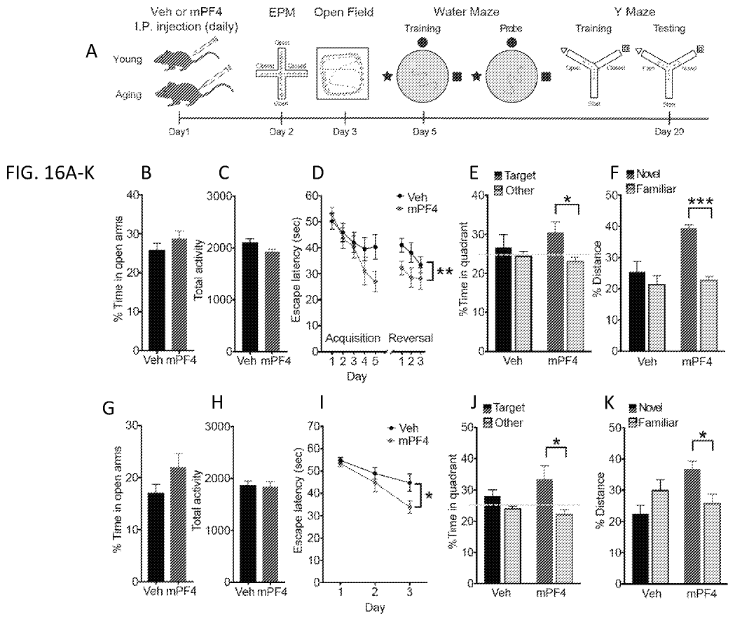

LTP underlies mechanisms of cognition, a highly valued manifestation of brain function. Therefore, we tested whether systemic PF4, like systemic klotho (10), can enhance learning and memory. Young adult mice (3-5 months) were treated daily with vehicle or systemic mouse PF4 (20 μg/kg i.p.) (FIG. 16A). PF4 did not alter anxiety-like behavior in the elevated plus maze (FIG. 16B) or hyperactivity in the open field (FIG. 16C). In the Morris watermaze, PF4 treatment augmented spatial learning during hidden platform training, during both acquisition and reversal of the platform location (FIG. 16D). In a probe trial, PF4 enhanced memory retention (FIG. 16E). Likewise, in the two-trial Y maze, PF4 increased exploration in the novel compared with familiar arm of the maze following context training, indicating that it enhanced spatial and working memory in young mice (FIG. 16F). Thus, PF4 specifically enhanced learning and memory in young adult mice, without altering other behaviors.

Since aging is the primary risk factor for cognitive impairment, we tested whether PF4 could enhance cognition in the aging brain. Aging mice (17-20 months) were treated daily with vehicle or systemic PF4 (20 μg/kg i.p.). Like in young mice, PF4 did not alter anxiety-like behavior (FIG. 16G) or hyperactivity (FIG. 16H). In tests of spatial and working memory, PF4 augmented spatial learning (FIG. 16I) and memory (FIG. 16J) in watermaze testing of aging mice. Further, it enhanced spatial and working memory in two-trial Y maze testing of aging mice (FIG. 16K). Thus, acute treatment with PF4 boosted cognition in the aging brain.

Our study reveals an unconventional role for platelets in enhancing cognition in the young and aging brain. Klotho induced platelet activation following a cognitive task in a manner similar to exercise-induced platelet activation (2). Since exercise increases klotho (23), it is interesting to speculate that each of their signaling pathways for platelet activation, yet to be determined, converges upon release of PF4. Our findings indicate that platelets can act as circulating messengers of klotho-mediated cognitive enhancement and modulate cognition itself through release of PF4. To this end, PF4 may engage glutamatergic signaling in the brain through mechanisms that remain to be determined. Augmenting platelet factors such as PF4 will enhance cognition and counteract cognitive deficits in the aging brain.
1. P. E. J. van der Meijden, J. W. M. Heemskerk, Platelet biology and functions: new concepts and clinical perspectives. Nat Rev Cardiol 16, 166-179 (2019).
2. O. Leiter et al., Exercise-Induced Activated Platelets Increase Adult Hippocampal Precursor Proliferation and Promote Neuronal Differentiation. Stem Cell Reports 12, 667-679 (2019).
3. O. Leiter, T. L. Walker, Platelets: The missing link between the blood and brain? Prog Neurobiol 183, 101695 (2019).
4. M. T. Chateau, C. Araiz, S. Descamps, S. Galas, Klotho interferes with a novel FGF-signalling pathway and insulin/Igf-like signalling to improve longevity and stress resistance in Caenorhabditis elegans. Aging 2, 567-581 (2010).
5. H. Kurosu et al., Suppression of aging in mice by the hormone Klotho. Science 309, 1829-1833 (2005).
6. A. P. Singh et al., alphaKlotho Regulates Age-Associated Vascular Calcification and Lifespan in Zebrafish. Cell Rep 28, 2767-2776 e2765 (2019).
7. D. B. Dubal et al., Life extension factor klotho enhances cognition. Cell Rep 7, 1065-1076 (2014).
8. D. B. Dubal et al., Life extension factor klotho prevents mortality and enhances cognition in hAPP transgenic mice. J Neurosci 35, 2358-2371 (2015).
9. A. M. Laszczyk et al., Klotho regulates postnatal neurogenesis and protects against age-related spatial memory loss. Neurobiol Aging 59, 41-54 (2017).
10. J. Leon et al., Peripheral Elevation of a Klotho Fragment Enhances Brain Function and Resilience in Young, Aging, and alpha-Synuclein Transgenic Mice. Cell Rep 20, 1360-1371 (2017).
11. A. Masso, A. Sanchez, A. Bosch, L. Gimenez-Llort, M. Chillon, Secreted alphaKlotho isoform protects against age-dependent memory deficits. Mol Psychiatry 23, 1937-1947 (2018).
12. Y. Zhao et al., Klotho overexpression improves amyloid-beta clearance and
cognition in the APP/PS1 mouse model of Alzheimer's disease. Aging Cell, e13239 (2020).
13. M. C. Hu et al., Renal Production, Uptake, and Handling of Circulating alphaKlotho. J Am Soc Nephrol 27, 79-90 (2016).
14. J. S. Yokoyama et al., Systemic klotho is associated with KLOTHO variation and predicts intrinsic cortical connectivity in healthy human aging. Brain Imaging Behav 11, 391-400 (2017).
15. J. S. Yokoyama et al., Variation in longevity gene KLOTHO is associated with greater cortical volumes. Ann Clin Transl Neurol 2, 215-230 (2015).
16. N. Maugeri et al., Platelet microparticles sustain autophagy-associated activation of neutrophils in systemic sclerosis. Sci Transl Med 10, (2018).
17. R. G. Morris, E. Anderson, G. S. Lynch, M. Baudry, Selective impairment of learning and blockade of long-term potentiation by an N-methyl-D-aspartate receptor antagonist, APS. Nature 319, 774-776 (1986).
18. K. Nakazawa, T. J. McHugh, M. A. Wilson, S. Tonegawa, NMDA receptors, place cells and hippocampal spatial memory. Nat. Rev. Neurosci. 5, 361-372 (2004).
19. M. P. Lambert et al., Platelet factor 4 is a negative autocrine in vivo regulator of megakaryopoiesis: clinical and therapeutic implications. Blood 110, 1153-1160 (2007).
20. D. E. Eslin et al., Transgenic mice studies demonstrate a role for platelet factor 4 in thrombosis: dissociation between anticoagulant and antithrombotic effect of heparin. Blood 104, 3173-3180 (2004).
21. W. Lu, J. Du, A. Goehring, E. Gouaux, Cryo-EM structures of the triheteromeric NMDA receptor and its allosteric modulation. Science 355 (2017).
22. P. Paoletti, J. Neyton, NMDA receptor subunits: Function and pharmacology. Current opinion in pharmacology 7, 39-47 (2007).
23. F. J. Amaro-Gahete et al., Role of Exercise on S-Klotho Protein Regulation: A Systematic Review. Curr Aging Sci 11, 100-107 (2018).

The above examples are provided to illustrate the invention but not to limit its scope. Other variants of the invention will be readily apparent to one of ordinary skill in the art and are encompassed by the appended claims. All publications, databases, internet sources, patents, patent applications, and accession numbers cited herein are hereby incorporated by reference in their entireties for all purposes.

What is claimed is:

1. A method for improving cognitive function in an individual in need thereof comprising
administering to the individual an effective amount of a protein comprising a polypeptide of Table 1 or Table 2 or a functional fragment or variant thereof, wherein the administering is systemic or peripheral,
thereby improving cognitive function in the individual.

2. The method of claim 1, wherein the polypeptide comprises Platelet Activating Factor 4 (PF4) or a functional fragment or variant thereof.

3. The method of claim 2, wherein the polypeptide comprises an amino acid sequence at least 70, 75, 80, 85, 90, 95, 97, or 99% identical to SEQ ID NO:1.

4. The method of claim 1, wherein the administering is oral, mucosal, or carried out by injection.

5. The method of claim 4, wherein the injection is intravenous, intraperitoneal, subcutaneous, or intramuscular.

6. The method of any one of the proceeding claims, wherein the individual is a human.

7. The method of claim 6, wherein the human has at least normal cognitive function and the administering results in improved cognitive function compared to before the administering.

8. The method of claim 6, wherein the human is 50 years of age or older.

9. The method of claim 8, wherein the human has age related cognitive decline.

10. The method of claim 6, wherein the human is less than 50 years of age.

11. The method of any one of the proceeding claims, wherein the individual is a human having a neurodegenerative disease.

12. The method of claim 11, wherein the neurodegenerative disease is selected from the group consisting of: Alzheimer's disease, Parkinson's disease, Huntington's disease, frontotemporal dementia, progressive supranuclear palsy, corticobasalar degeneration, mild cognitive impairment, vascular dementia, Lewy body dementia, multiple system atrophy, amyotropic lateral sclerosis, prion disorder, and HIV-related dementia.

13. The method of any one of the foregoing claims, wherein the individual is a human having a condition selected from the group consisting of: depression, schizophrenia, attention deficit/hyperactivity disorder, autism spectrum disorder, intellectual disability, a mood disorder, and a psychotic disorder.

14. The method of any one of the foregoing claims, wherein the individual is a human having a condition selected from the group consisting of traumatic brain injury, stroke, multiple sclerosis, neuroautoimmune disease, epilepsy, delirium, and a paraneoplastic disorder.

15. The method of any one of the foregoing claims, wherein the individual is a human having a condition selected from the group consisting of: an X-linked mental disorder, Down's syndrome, Angelman's syndrome, Rett's syndrome, phenylketonuria, Lesch-Nyhan, galactosemia, and adrenoleukodystrophy.

16. The method of any one of the foregoing claims, wherein the individual is a human having a condition selected from astrocytoma, ependymoma, medulloblastoma, and oligodendroglioma.

17. The method of any one of the foregoing claims, wherein the individual is a human receiving radiation treatment or chemotherapy for cancer.

18. The method of any one of the foregoing claims, wherein the individual is a human that is experiencing, or will experience within 24 hours, sleep deprivation or jet lag.

19. The method of any one of the foregoing claims, wherein the effective amount is 1 μg to 1000 μg per kg body weight of the individual.

20. The method of any one of the foregoing claims, wherein the polypeptide or a functional fragment thereof is administered more than once as part of a course of treatment.

21. The method of claim 20, wherein the polypeptide or a functional fragment thereof is administered once every 1-7 days.

22. The method of any one of the foregoing claims, further comprising testing the cognitive function of the individual after administering.

23. The method of claim 22, further comprising testing the cognitive function of the individual prior to administering, and comparing the cognitive function of the individual prior to and after administering.

24. The method of claim 22 or 23, wherein cognitive function is determined by testing the individual for semantic, episodic, procedural, priming, and/or working memory.

25. A method for improving motor function or motor learning or both in an individual in need thereof comprising
administering to the individual an effective amount of a protein comprising a polypeptide of Table 1 or Table 2 or a functional fragment or variant thereof, wherein the administering is systemic or peripheral,
thereby improving motor function in the individual compared to before the administering.
